(19) United States
(12) Patent Application Publication
(10) Pub. No.: US 2005/0170359 A1
(43) Pub. Date: Aug. 4, 2005

(54) Treatment of vascular dysfunction and alzheimer's disease

(76) Inventor: Berislav V. Zlokovic, Rochester, NY (US)

Correspondence Address: NIXON & VANDERHYE, PC, 901 NORTH GLEBE ROAD, 11TH FLOOR, ARLINGTON, VA, 22203, US

(21) Appl. No.: 10/516,729

(22) PCT Filed: Jun. 11, 2003
(86) PCT No.: PCT/US03/18334

Related U.S. Application Data
(60) Provisional application No. 60/387,426, filed on Jun. 11, 2002.
(60) Provisional application No. 60/387,427, filed on Jun. 11, 2002.
(60) Provisional application No. 60/387,913, filed on Jun. 13, 2002.

(51) Int. Cl. C12Q 1/68; G01N 33/53; G01N 33/567; A01K 67/00; C12N 5/06

(52) U.S. Cl. 435/6; 435/7.2; 800/12; 435/354

(57) ABSTRACT

Dysregulation of vascular function is observed in brain endothelium derived from patients affected by Alzheimer's disease. This may be manifested at the cell or organ level: e.g., abnormal capillary morphogenesis, defective angiogenesis, inappropriate cellular senescence, mitotic catastrophe, or combinations thereof. This observation can be used for diagnosis or treatment of neurodegenerative disorders and other cognitive impairments.

### CROSS-REFERENCE TO RELATED APPLICATIONS

This application claims the benefit of provisional Appln. No. 60/387,426, filed Jun. 11, 2003; Appln. No. 60/387,427, filed Jun. 11, 2003; and Appln. No. 60/387,913, filed June 13, 2003. The contents of these provisional patent applications and Appln. No. PCT/US02/01069 are incorporated herein by reference.

### FIELD OF THE INVENTION

The invention relates to neurodegenerative disorders and cognitive impairments (e.g., Alzheimer's disease) and the dysregulation of vascular function which is observed in brain endothelial cells (BEC) derived from patients.

### BACKGROUND OF THE INVENTION

Brain degenerative diseases are associated with dysfunction of learning, memory, and/or cognition include cerebral senility, multi-infarct dementia, senile dementia of the Alzheimer type, age-associated memory impairment, and certain disorders associated with Parkinson's disease. Alzheimer's disease is the most common of the age-related neurodegenerative diseases: between about 10% and 20% of individuals over age 70 are affected, and about 50% of those over age 85 are affected. It is estimated that about 50% of nursing home residents in the U.S. are affected, and that the annual costs associated with the care of patients with Alzheimer's disease in this country are in excess of $65 billion. As the population ages, the prevalence of Alzheimer's disease will increase dramatically from four million presently in the U.S. to more than 10 million by 2015. Study of the molecular basis of Alzheimer's disease complements behavioral studies. It can lead to a better understanding of pathogenesis and mechanisms of disease, as well as new modes of treatment.

Current dogma teaches that many different initiating events will ultimately cause synapses to fail to function properly and this leads inexorably to neuronal death. Several neuropathological findings are associated with Alzheimer's disease and the following are considered indicia of the.Alzheimer's phenotype: intraneuronal deposits of neurofibrillary tangles (NFT), parenchymal amyloid deposits—neuritic plaques, cerebral amyloid angiopathy (CAA), and synaptic loss. Popular current theories for the cause of Alzheimer's disease are the amyloid, tau, and inflammatory theories. Mutations in three genes encoding amyloid-β precursor protein (APP), presenilin-1, and presenilin-2 cause rare, early-onset, autosomal dominant forms of Alzheimer's disease. These mutations all affect APP metabolism such that more amyloid-β (AP) peptide is produced. In contrast, most cases of Alzheimer's disease have ages of onset above 65 years and exhibit no clear pattern of inheritance (i.e., late onset Alzheimer's disease or LOAD). The E4 allele of the apolipoprotein E (apoE) gene is the only known risk factor for LOAD. But 50% of late-onset cases carry no apoE4 alleles, which indicates that there must be additional risk factors. Recent studies have identified the locus for LOAD on chromosome 10 and linked it with increased levels of circulating Aβ₁₋₄₂. See refs. 1-10.

Deposition of Aβ in the CNS occurs during normal aging and is accelerated by Alzheimer's disease. Aβ is implicated in the neuropathology of Alzheimer's disease and related disorders. Recent studies suggest that the BBB plays a major role in determining the concentration of Aβ in the CNS. The BEC at the BBB have a dual role: (a) to control entry of plasma-derived Aβ and its binding/transport proteins into the CNS, and (b) to regulate levels of brain-derived Aβ via clearance mechanisms. See refs. 11-22.

Previous genetic and biochemical approaches neither taught nor suggested that Alzheimer's disease is associated with or may be caused by disease-specific mRNA and protein profiles in BEC related to altered BEC biology distinct from normal aging, that could be manifested at the organ level as abnormal responses to angiogenic stimulation, aberrant formation of brain capillaries, formation of incompetent brain capillary networks, accelerated and/or premature removal of BEC from the vascular system during capillary morphogenesis through cell death programs (e.g., apoptosis, aniokis, mitotic catastrophe), and development of a senescent phenotype resulting in loss of regulatory functions of the BBB and CBF (cf. St. George-Hyslop, Sci. Am. pp. 76-83, December 2000). In particular, it was only recently described that dysfunction of brain endothelium may cause and/or be the result of disease (Int'l Patent Appln. PCT/US02/01069).

These observations can be used to improve our understanding of the pathogenesis of Alzheimer's disease and mechanisms of disease. Novel and inventive methods of diagnosis and treatment are suggested by them. Other advantages of the invention are discussed below or would be apparent from the disclosure herein.

References

1. Wisniewski et al. (1997) Biology of Aβ amyloid in Alzheimer's disease. Neurobiol. Dis. 4:311-328.

2. Selkoe (1997) Alzheimer's disease: Genotype, phenotype, and treatments. Science 275:630631.

3. Selkoe (1998) The cell biology of beta-amyloid precursor protein and presenilin in Alzheimer's disease. Trends Cell Biol. 8:447453.

4. Younkin (1998) The role of A beta 42 in Alzheimer's disease. J. Physiol. (Paris) 92:289-292.

5. Roses (1998) Alzheimer disease: A model of gene mutations and susceptibility polymorphisms for complex psychiatric diseases. Amer. J. Med. Gen. 81:49-57.

6. Hardy et al. (1998) Genetic dissection of Alzheimer's disease and related dementias: Amyloid and its relationship to tau. Nature Neurosci. 1:355-358.

7. Dickson (1997) The pathogenesis of senile plaques. J. Neuropathol. Exp. Neurol. 56:321-339.

8. Blacker et al. (1998) Alpha-2 macroglobulin is genetically associated with Alzheimer disease. Nature Gen. 19:357-360.

9. Ertekin-Taner et al. (2000) Linkage of plasma Aβ42 to a quantitative locus on chromosome 10 in late-onset Alzheimer's disease pedigrees. Science 290:2303-2304.

10. Myers et al. (2000) Susceptibility locus for Alzheimer's disease on chromosome 10. Science 290:2304-2305.

11. Zlokovic (1997) Can blood-brain barrier play a role in the development of cerebral amyloidosis and Alzheimer's disease pathology. Neurobiol. Dis. 4:23-26.

12. Zlokovic et al. (1993) Blood-brain barrier transport of circulating Alzheimer's amyloid β. Biochem. Biophys. Res. Commun. 197:1034-1040.

13. Maness etal. (1994) Passage of human amyloid-β protein 1-40 across the murine blood-brain barrier. Life Sci. 55:1643-1650.

14. Poduslo et al. (1997) Permeability and residual plasma volume of human, Dutch variant, and rat amyloid β-protein 1-40 at the blood-brain barrier. Neurobiol. Dis. 4:27-34.

15. Ghilardi et al. (1996) Intra-arterial infusion of [¹²⁵I]Aβ 1-40 labels amyloid deposits in the aged primate brain in vivo. Neuroreport 7:2607-2611.

16. Mackic et al. (1998) Cerebrovascular accumulation and increased blood-brain barrier permeability to circulating Alzheimer's amyloid-β peptide in aged squirrel monkey with cerebral amyloid angiopathy. J. Neurochem. 70:210-215.

17. Zlokovic et al. (1996) Glycoprotein 330/megalin: Probable role in receptor-mediated transport of apolipoprotein J alone and in a complex with Alzheimer's disease amyloid β at the blood-brain and blood-cerebrospinal fluid barriers. Proc. Natl. Acad. Sci. USA 93:4229-4236.

18. Martel et al. (1997) Isoform-specific effects of apolipoproteins E2, E3, E4 on cerebral capillary sequestration and blood-brain barrier transport of circulating Alzheimer's amyloid β. J. Neurochem. 69:1995-2004.

19. Shibata et al. (2000) Clearance of Alzheimer's amyloid-beta 1-40 peptide from brain by low-density lipoprotein receptor-related protein-1 at the blood brain barrier. J. Clin. Invest. 106:1489-1499.

20. Mackic et al. (1998) Human blood-brain barrier receptors for Alzheimer's amyloid-β 1-40: Asymmetrical binding, endocytosis and transcytosis at the apical side of brain microvascular endothelial cell monolayer. J. Clin. Invest. 102:734-743.

21. Zlokovic (1996) Cerebrovascular transport of Alzheimer's amyloid-β and apolipoproteins J and E: Possible anti-amyloidogenic role of the blood-brain barrier. Life Sci. 59:1483-1497.

22. Ghersi-Egea et al. (1996) Fate of cerebrospinal fluid-borne amyloid β-peptide: Rapid clearance into blood and appreciable accumulation by cerebral arteries. J. Neurochem. 67:880-883.

### SUMMARY OF THE INVENTION

In one embodiment of the invention, reagents are provided in kit form that can be used for performing the methods such as the following: diagnosis, identification of those at risk for disease or already affected, or determination of stage of disease or its progression. In addition, the reagents may be used in methods related to the treatment of disease such as the following: evaluation whether or not it is desirable to intervene in the disease's natural history, alteration of the course of disease, early intervention to halt or slow progression, promotion of recovery or maintenance of function, provision of targets for beneficial therapy or prophylaxis, comparison of candidate drug, medical, or surgical regimens, or determination of the effectiveness of a drug, medical, or surgical regimen. The instructions for performing these methods, reference values and positive/negative controls, and relational databases containing patient information (e.g., genotype, medical history, symptoms, transcription or translation yields from gene expression, physiological or pathological findings) are other products considered to be aspects of the invention.

In other embodiments of the invention, the methods for diagnosis and treatment are provided. For screening of drugs and clinical trials, the respective drug and medical/surgical regimen selected are also considered to be embodiments of the invention. The amount and length of treatment administered to a cell, tissue, or individual in need of therapy or prophylaxis is effective in treating the affected cell, tissue, or individual. One or more properties/functions of affected endothelium or cells thereof, or the number/severity of symptoms of affected individuals, may be improved, reduced, normalized, ameliorated, or otherwise successfully treated. The invention may be used alone or in combination with other known methods. Instructions for performing these methods, reference values and positive/negative controls, and relational databases containing patient information are considered further aspects of the invention. The individual may be any animal or human. Mammals, especially humans and rodent or primate models of disease, may be treated; thus, both human and veterinary treatments are contemplated.

Further aspects of the invention will be apparent to a person skilled in the art from the following detailed description and claims, and generalizations thereto.

### DESCRIPTION OF THE TABLES AND DRAWINGS

FIG. 1 shows brain capillary morphogenesis mediated by control and AD BEC in 3-D collagen matrices after stimulation with VEGF/bFGF (40 ng/ml) using an assay system as reported (Davis et al., J. Cell Sci. 114:917-930, 2001). FIGS. 1A-1C show formation of brain capillaries from age-matched control BEC: (A) the formation of intracellular vacuoles at 4 hr (arrow, bar 20 μm); (B) vacuoles at 15 hr (arrows, toluidine staining, bar 25 μm); and (C) capillary tubes at 24 hr (arrows, Hoechst staining, bar 12 μm). FIGS. 1D-1F show aberrant brain capillary formation mediated by AD BEC: (D) apoptotic bodies (arrow), condensed chromatin, and/or 10 fragmented nuclei (asterisk, Hoechst, bar 12 μm); (E) blebbing of the cytoplasmic membrane (bar 12 μm); and (F) nuclear fragmentation (asterisk; Hoechst, 12 μm) and apoptotic bodies (arrows) at 24 hr.

FIG. 2 summarizes quantitative data on time course of formation of intracellular vacuoles (stage I) and tubes (stage II) during brain capillary morphogenesis mediated by AD or control BEC after stimulation with VEGF/bFGF as in FIG. 1. FIG. 2A shows the percentage of cells forming vacuoles was calculated as a fraction of total number of cells. FIG. 2B shows the number of tubes and FIG. 2C shows the total tube length determined at 24 hr. Values are mean±s.e. from 12 to 20 measurements derived from six cases for each studied group; each case was tested in triplicate or greater. Significance was evaluated by Student's t-test.

FIG. 3 shows apoptosis during BEC differentiation into brain capillary tubes in AD model studied in 3-D collagen gel cultures. TUNEL positive cells (bar=15 μm) with nuclear changes (Hoechst) are seen in AD BEC after stimulation with VEGF/bFGF at 4 hr (FIGS. 3A-3B); no such changes were observed in age-matched BEC (FIGS. 3C-3D). FIG. 3E shows the number of TUNEL positive cells at 4 hr. Mean±s.e., from 6 cases per group determined for each case in 8 to 10 different fields; *p<0.01, AD vs. controls by Student t-test. Western blot analyses of cell lysates for p53 and activated form of caspase 3 at 4 hr (FIG. 3F), 12 hr (FIG. 3G), and 24 hr (FIG. 3H) during brain capillary morphogenesis. Relative abundance of p53 (FIG. 3I) and caspase-3 (FIG. 3J) normalized to β-actin in young and age-matched controls and AD. The relative levels of expression of p53 and caspase-3 in young controls were arbitrarily set as 1 for each studied time point. Mean±s.e., from 4 to 5 cases; *p<0.01, AD vs. controls by Student t-test. VEGF and bFGF, 40 ng/ml.

FIG. 4 shows that zVAD, a broad caspase-3 inhibitor, restores tube formation during AD BEC-mediated brain capillary angiogenesis. zVAD-fmk (50 μM) reduced the levels of activated form of caspase-3 in AD BEC at 24 hr (FIG. 4A), and restored the number of tubes (FIG. 4B) and total tube length (FIG. 4C) during AD BEC-mediated morphogenesis. Mean±s.e., from 4 cases per group; significance by Student t test. VEGF and bFGF, 40 ng/ml.

FIG. 5 shows increased expression of p53 and caspase 3 in brain microvessels in situ in patients with AD compared to age-matched controls, thus corroborating our findings in BEC model (FIG. 3). Double staining for collagen IV (vascular basement membrane marker) and p53 in Brodmann's areas 9 and 10 in AD (FIGS. 5A-5B) or age-matched controls (FIGS. 5C-5D) was performed. Double staining for collagen IV and the active form of caspase-3 in Brodmann's areas 9 and 10 in AD patients (FIGS. 5E-5F) or age-matched controls (FIGS. 5G-5H) was performed. The number of p53-positive vessels (FIG. 5I) and caspase 3-positive vessels (FIG. 5J) relative to collagen IV in areas 9 and 10 in AD and age-matched controls was calculated. Mean±s.e., n=6 cases per group.

FIG. 6 shows that activated protein C (APC), recently shown to exhibit significant anti-apoptoic activity in BEC during hypoxia/ischemia (Cheng et al., Nature Med. 9:338-342, 2003), improves tubule formation, prevents apoptosis of AD BEC, and enhances their migration. AD or age-matched control (AMC) BEC (2×10⁴ cells/well) were plated in 2-D collagen matrigels (growth factors per BD Biosciences, Bedford, Mass.) and treated with 100 nM APC or vehicle (−APC). Total tube length was determined at 24 hr (FIG. 6A). Mean±s.e., n=30 to 40 assays per group from four AD and three AMC cases. AD or AMC BEC were treated with 100 nM APC, 100 nM boiled (B) APC, 100 nM serine mutant (M) APC, or vehicle (−APC) for 24 hr. Total tube length was determined at 24 hr (FIG. 6B). Mean±s.e., n=10 assays per group from one AD and one AMC case. % TUNEL-positive BEC at 4 hr during AD-mediated or AMC-mediated capillary morphogenesis in the presence of 100 nM APC or vehicle (−APC) was calculated (FIG. 6C). Mean±s.e., n=20 assays per group from two AD and two AMC cases. % Caspase-3-positive BEC at 4 hr of brain capillary morphogenesis in AD or AMC treated with 100 nM APC or vehicle (−APC) was calculated (FIG. 6D). Mean±s.e., n=20 assays per group from two AD and two AMC cases. AD BEC (3×10⁴) cells were assayed for migration in a modified Boyden chamber in the presence of vehicle (−APC), 100 nM APC, anti-APC C3 monoclonal antibody (200 μg/ml), or anti-APC+APC after 6 hr of incubation (FIG. 6E). Mean±s.e., n=6 assays per group from two AD cases. Migration of AD BEC was also assayed in the presence of vehicle (−APC), 100 nM APC, anti-EPCR antibody raised against the APC binding site (Dr Fukudome; 200 μg/ml), or anti-EPCR+APC (FIG. 6F). Mean±s.e., n=6 assays per group from two AD cases.

FIG. 7 shows impaired cell growth of AD BEC compared to age-matched control brains and the presence of enlarged β-galactosidase positive cells with flattened morphology. FIG. 7A shows growth curves for AD BEC (n=3) and age-matched control (AMC) BEC (n=4). The culture was maintained subconfluent for 5 days. The cells in triplicate wells were counted daily (mean±s.e.m.). Population doubling time (PDT) in AD was compared to AMC (p<0.01) in FIG. 7B. Accelerated senescence of AD BEC is shown by senescence-associated β-galactosidase (<-gal) assay of cultured BEC for AD at PD 23 (FIG. 7C) and PD 34 (FIG. 7D), and AMC at PD 44 (FIG. 7E). Bar=50 μm. Total replicative potential was expressed as CPDL (cumulative population doubling life) in AD BEC (n=8) compared in FIG. 7F to AMC BEC (n=5).

FIG. 8 shows the H₂O₂ model of stress-induced premature senescence (SIPS) in young human BEC that result in a cellular phenotype similar to that seen in replicative BEC senescence of AD. Subconfluent BEC from young humans were treated with 300 μM of H₂O₂ for 2 hr followed by subculture. Growth arrest in untreated control BEC (FIG. 8A) or SIPS BEC (FIG. 8B) was determined by BrdU incorporation and FACS analysis. Morphological change and SA-β-galactosidase activity typical for senescent phenotype five days after H₂O₂ treatment was shown in untreated control BEC (FIG. 8C) or SIPS BEC (FIG. 8D). Bar=50 μm. Phosphorylation of p38 and p53, and induction of p53 and p21 in response to H₂O₂ was determined by Western blot analyses (FIG. 8E, time in minutes). Activation of p53, p21, and p16 over 5 days after H₂O₂-treatment was shown by Western blot analyses (FIG. 8F, time in days).

FIG. 9 shows upregulation of p16 during replicative or stress-induced senescence in AD BEC cultures. Western blot analysis of p16 in AD BEC cultures from passage 5-7 (FIG. 9A, case numbers are indicated above passage numbers) and signal intensity for p16 normalized for β-actin (FIG. 9C, n=4) are shown. H₂O₂ stress-induced senescence increases p16 over six days (FIG. 9B, case numbers are indicated above time in days) and signal intensity normalized for β-actin (FIG. 9D, n=5) are shown.

FIG. 10 confirms increased number of p16-positive brain microvessels in AD (A-C) compared to age-matched controls (D-F), thus corroborating findings in the in vitro BEC model. Brain sections from Brodmann's area 9 or 10 were stained with antibodies to Von Willebrand factor (FIGS. 10A and 10D) and p16 (FIGS. 10D and 10E) as well as merged images (FIGS. 10C and 10F). The number of p16-positive microvessels was scored for three AD and three AMC cases (FIG. 10G, bar=50 μm).

FIG. 11 shows that p38 MAPK inhibitor SB202190 prevents BEC-mediated aberrant angiogenesis caused by either stress-induced premature senescence (SIPS) in control cells or in AD BEC. In 3D collagen gels (as described in FIG. 1), BEC that reached greater than 90% replicative senescence (RS) or were exposed to H₂O₂ (SIPS) formed an insignificant number of tubes (FIGS. 11A-11B, bar=50 μm). Total tube length—control BEC (arbitrarily taken as 100%) were compared to RS and SIPS BEC (FIG. 11C). The effect of SB202190 (SB, 10 μM for 1 hr) on SIPS-induced aberrant angiogenesis. Mean±s.e., n=9 assays per group from three different cases/group. In 2D collagen matrigels (growth factors per BD Biosciences, Bedford, Mass.), SB202190 (SB, 10 μM) increased the number of tubes in early passage AD BEC (FIGS. 11D-11E, bar=50 μm). SB202190 (SB, 10 μM) increases total tube length in both AMC and AD BEC (FIG. 11F). Mean±s.e., n=6-9 assays per group from two AMC and three AD cases.

Tables 1-8 summarize characteristics of patients and controls used for different types of studies including gene expression profiling on Affymetrix U95A, U133A, and U133B chips, and for senescence studies.

Tables 9-12 summarize changes in gene expression in BEC AD vs. age-matched controls using Affymetrix U95A, U133A, and U133B chips, and coincidence analysis of genes in RS-AD and SIPS-YC suggesting that SIPS model has significant features of BEC senescence AD-type.

### DETAILED DESCRIPTION OF THE INVENTION

The dysregulation of vascular function which is observed in brain endothelial cells (BEC) derived from patients with Alzheimer's disease (AD) is not related to previously observed pathology like that involved in production of amyloid (i.e., synthesis of amyloid precursor protein and its processing and metabolism). Such dysregulation may be manifested at the cell or organ level as abnormal BEC differentiation in response to angiogenic signaling, activation of a programmed cell death through apoptosis and/or other forms of death (e.g., anoikis, mitotic catastrophe) during brain capillary morphogenesis, development of premature cellular senescence, or combinations thereof. The altered biology of BEC derived from AD patients is associated with, or contributes to and/or may result from a disease-specific gene expression profiles associated with changes in expression of genes with predicted actions in cell differentiation, angiogenesis, signal transduction, cytoskeleton, matrix, lipid metabolism, etc., largely independent of normal aging. These related molecular abnormalities in AD BEC ultimately lead to a failure of successful brain vascular repair in AD (e.g., aberrant formation of new incompetent brain capillaries) and/or vascular senescence with altered BEC phenotype and greatly diminished brain capillary functions. These observations can be used to diagnose a vascular disorder in AD that clinically may present as abnormalities in cerebral blood flow (CBF) and blood-brain barrier (BBB) regulatory functions associated with mild cognitive impairment (MCI) in asymptomatic individuals or dementia in symptomatic individuals, to identify those at risk for the vascular disease AD type or those already affected thereby, to determine the stage of the disease or the disease's progression, to intervene earlier in or alter the disease's natural history, to provide targets for therapeutic or prophylactic treatments, to screen drugs or compare medical regimens, to determine the effectiveness of a drug or medical regimen in treating the disease, or any combination thereof. Examples of drug-based therapies for AD vascular disorder involving small molecules and proteins are reported.

These studies are distinguished from previous neural and vascular theories for explaining the etiology and pathogenesis of Alzheimer's disease because they focus on the roles of BEC differentiation, brain vascular repair, and abnormal angiogenesis, on the one hand, and cellular senescence of BEC, on the other, in promoting dysfunction of the vascular endothelium and vascular disorder in Alzheimer's disease. Furthermore, these studies link for the first time a subset of disease-specific genes (e.g., 0.3% of 12,500 genes using Affymetrix U95A chips; 0.2-0.3% of 45,000 genes using Affymetrix U133A and U133B chips) to abnormalities in AD BEC biology as demonstrated by an in vitro AD BEC model and corroborated by the analysis of brain vessels in AD tissue in situ. Endothelial cells of brain microvessels, which are derived mainly from capillaries (about 90% to 95%) and a small percentage (about 5 to 10%) originating from smaller venuies and arterioles (less than 20 μm diameter), have been studied. A role for dysregulation of vascular function is demonstrated herein which differs from the previous vascular theories of AD, centered on changes in circulating Aβ transport through the BBB: e.g., apoj-, apoE-, RAGE- (Zlokovic et al., Proc. Natl. Acad. Sci. USA 93:4229-4236, 1996; Martel et al., J. Neurochem. 69:1995-2004, 1997; Mackic et al., J. Clin. Invest. 102:734-743, 1998; Deane et al., Nature Med., in press); and/or LRP-1-mediated clearance of Aβ from the brain (Zlokovic et al., Nature Med. 6:718-719, 2000; Shibata et al., J. Clin. Invest. 106:1489-1499, 2000; Zlokovic et al., In: Aβ Metabolism in Alzheimer's Disease, Ed. T. Saido, Landes Bioscience, pp. 114-122, 2003), clearance by peripheral Aβ-plasma binding agents including anti-Aβ antibodies (DeMattos et al., Science 295:2264-2267, 2002), gelsolin and gangli-oside M2 (Matsuoka et al., J. Neurosci. 23:29-33, 2003), sRAGE (Deane et al., Nature Med., in press), sLRP-1 clusters 11 and IV (Zlokovic et al. Soc. Neurosci. Abstract No. 1974, in press) and/or permeabilizers of the BBB such as insulin-like growth factor-1 (Carro et al., Nature Med. 8:1390-1397, 2002); association of amyloid with blood vessels and early onset familial form of cerebral amyloid angiopathy (CAA) (Levy et al., Science 248:1124-1126, 1990; Van Broeckhoven et al., Science 248, 1120-1122, 1990; Haass et. al., J. Biol. Chem. 269:17741-17748, 1994; Grabowski et al., Ann. Neurol. 49:697-705, 2001; De Jonghe et al., Neurobiol. Dis. 5:281-286, 1998; Hendricks et al., Nature Genet. 1:218-221, 1992; Kamino etal., Am. J. Hum. Genet. 51:998-1014, 1992; Tagliavini et al., Alzheimer's Reports 2, S28, 1999); vasoconstrictory effects of Aβ on blood vessels and proinflammatory and vasoactive effects of Aβ in the cerebrovasculature (Thomas et al., Nature 380:168-171, 1996), Paris et al., Neurobiol. Aging 21, 183-197, 2000; Townsend et al., Ann. NY Acad. Sci. 977:65-76, 2002; Volmar et al., Soc. Neurosci. Abstract No. 882.1, 2002); cerebral endothelial dysfunction in mice overexpressing Aβ precursor protein that can be rescued by reactive oxygen species scavengers (ladecola et al., Nature Neurosci. 2:157-161, 1999); Aβ-related functional hyperemia and changes in CBF in Alzheimer's mouse model (Niwa et al., Proc. Natl. Acad. Sci. USA 97:9735-9740, 2000; Niwa et al., Neurobiol. Dis. 9:6168, 2002); brain capillary distortions and microvascular aberrations detected in brains of individuals with Alzheimer's disease by light or electron microscopy (Miyakawa et al., Virchows Arch. 40:121-129, 1982; Yamada et al., Dement. Geriatr. Cogn. Dis. 8:163-168, 1997, Grammas et al., J. Alzheimer's Dis. 4:217-223, 2002); reported risks for Alzheimer's disease compiled from epidemiological studies of elderly patients with reduced cerebral perfusion and/or risk factors associated with both Alzheimer's disease and vascular dementia (Breteler et al., Neurobiol. Aging 21:153-160, 2000; Hofman etal., Lancet 349:151-154, 1997; see for review de la Torre, Stroke 33:1152-1162, 2002; Zlokovic, Adv. Drug Deliv. Rev. 54:1533-1537, 2002; Zlokovic, Adv. Drug Deliv. Rev. 54:1553-1559, 2002; Rotterdam Scan Study, New Engl. J. Med. 348:1215-1222, 2003).

Endothelial cells and cultures thereof from brain (e.g., brain microvasculature, leptomeningeal vessels) or possibly other organs (e.g., bone marrow, blood containing endothelial precursor cells, extracranial blood vessels, skin) may be prepared from individuals at risk for Alzheimer's disease, affected by the disease, or not. Tissue may be obtained as biopsy or autopsy material; cells of interest may be isolated therefrom and then cultured. Also provided are extracts of cells (e.g., cytoplasm, membrane); at least partially purified nucleic acid and protein therefrom; and methods for their isolation. These reagents can be used to establish detection limits for assays, absolute amounts of gene expression that are indicative of disease or not, ratios of gene expression that are indicative of disease or not, and the significance of differences in such values. These values for positive and/or negative controls can be measured at the time of assay, before an assay, after an assay, or any combination thereof. Values may be recorded on storage medium and manipulated with computer software; storage in a database allows retrospective or prospective study. For example, the database may be physically stored on a tangible media like note paper or plastic transparency, mechanical switch or electronic valve, iron core, semiconductor RAM or ROM, magnetic or optical disk, or paper or magnetic tape. The medium may be erased, refreshed (e.g., dynamic), or permanent (e.g., static); it may be fixed or transportable. Information may be displayed or projected on a screen (e.g., tangible media such as a cathode ray tube, light emitting diode array, liquid crystal display). Genes that are increased, decreased, or not significantly changed in BEC are identified and related to the altered cell biology in Alzheimer's disease, and compared with the proprietary gene array data base, the cell biology data base, and the neuropathology and clinical data bases.

The reliability of diagnosis methods may be improved by (1) decreasing the incidence of false positive and false negatives and (2) increasing the sensitivity of detection. For example, the number of different genes that have a measurable difference in expression (i.e., increased or decreased) including a subset of disease-specific genes may be at least about 5, 10, 15, 20, 25, 30, 35, 40, 45, 50, 60, 70, 80, 90, 100, 120, 140, 160, 180, 190, 200, 250, 300, 350, 400, 450, 500, 600, 700, 800, or intermediate ranges thereof. The amount of change that is considered significant may be at least about 1.5-fold, 2-fold, 2.5-fold, 3-fold, 3.5-fold, 4-fold, 4.5-fold, 5-fold, 6-fold, 7-fold, 8-fold, 9-fold, 10-fold, 12-fold, 14-fold, 16-fold, 18-fold, 20-fold, or intermediate ranges thereof depending on Bayesian analysis. When expression is barely or even not detectable, the calculated ratio may be high and is not necessarily meaningful. The assay is quantitative in the sense that there is a direct and measurable relationship between the detected signal and gene expression (e.g., the number of transcripts or proteins), but the relationship does not necessarily need to be linear. In addition, a subset of disease-specific genes could be used to generate diagnostics arrays for AD vascular disorder.

Polynucleotides representative of genes that are increased or decreased in Alzheimer's disease may be used to identify, isolate, or detect complementary polynucleotides by binding assays. Similarly, polypeptides representative of the gene products that are increased or decreased in Alzheimer's disease may be used to identify, isolate, or detect interacting proteins by binding assays. Optionally, bound complexes including interacting proteins may be identified, isolated, or detected indirectly though a specific binding molecule (e.g., antibody, natural or nonnatural peptide mimetic) for the gene product that is increased or decreased in Alzheimer's disease. Interacting proteins may also be associated with or cause Alzheimer's disease. Affinity chromatography of DNA-binding proteins, electrophoretic mobility shift assay (EMSA), one- or two-hybrid system, membrane protein cross-linking, and screening a phage display library are techniques for identifying, isolating, or detecting interacting proteins.

Candidate compounds useful for treating Alzheimer's disease may interact with a representative polynucleotide or polypeptide, and be screened for their ability to provide therapy or prophylaxis. These products may be used in assays (e.g., diagnosis) or for treatment; conveniently, they are packaged as assay kits or in pharmaceutical form. Examples of drugs that are able to control aberrant AD BEC-mediated angiogenesis and/or prevent premature and/or accelerated apoptosis during AD BEC-mediated brain capillary morphogenesis (i.e., to reverse the dysfunctional vascular phenotype) are presented below.

Assaying Polynucleotides or Polypeptides

Binding of polynucleotides or polypeptides may take place in solution or on a substrate. The assay format may or may not require separation of bound from not bound. Detectable signals may be direct or indirect, attached to any part of a bound complex, measured competitively, amplified, or any combination thereof. A blocking or washing step may be interposed to improve sensitivity and/or specificity. Attachment of a polynucleotide or polypeptide, interacting protein, or specific binding molecule to a substrate before, after, or during binding results in capture of an unattached species. See U.S. Pat. Nos. 5,143,854 and 5,412,087.

Polynucleotide, polypeptide, or specific binding molecule may be attached to a substrate. The substrate may be solid or porous and it may be formed as a sheet, bead, fiber, tape, tube, or wire. The substrate may be made of cotton, silk, or wool; cellulose, nitrocellulose, nylon, or positively-charged nylon; natural, butyl, silicone, or styrenebutadiene rubber; agarose or polyacrylamide; crystalline silicon or polymerized organosiloxane; crystalline, amorphous, or impure silica (e.g., quartz) or silicate (e.g., glass); polyacrylonitrile, polycarbonate, polyethylene, polymethyl methacrylate, polymethylpentene, polypropylene, polystyrene, polysulfone, polytetrafluoroethylene, polyvinylidenefluoride, polyvinyl acetate, polyvinyl chloride, or polyvinyl pyrrolidone; or combinations thereof. Optically-transparent materials are preferred so that binding can be monitored and signal transmitted by light. For example, a bead suspended in solution and at the end of an optical fiber can be interrogated by a light signal (e.g., blue, red, or green) sent through the optical fiber when an analyte in solution (e.g., probe conjugated to a blue, red, or green label) binds to the bead, which is attached to the polynucleotide, polypeptide, or specific binding molecule.

Such reagents would allow capture of a molecule in solution by specific binding, and then interaction of the molecule with and immobilization to the substrate. Monitoring gene expression is facilitated by using an ordered substrate array or coded library of multiple substrates.

Polynucleotide, polypeptide, or specific binding molecule may be synthesized in situ by solid-phase chemistry or photolithography to directly attach the nucleotides or amino acids to the substrate. Attachment of the polynucleotide, polypeptide, or specific binding molecule to the substrate may be through a reactive group as, for example, a carboxy, amino, or hydroxy radical; attachment may also be accomplished after contact printing, spotting with a pin, pipetting with a pen, or spraying with a nozzle directly onto a substrate. Alternatively, the polynucleotide, polypeptide, or specific binding molecule may be reversibly attached to the substrate by interaction of a specific binding pair (e.g., antibody-digoxygenin/hapten/peptide epitope, biotin-avidin/streptavidin, glutathione S transferase or GST-glutathione, lectin-sugar, maltose binding protein-maltose, polyhistidine-nickel, protein A/G-immunoglobulin); cross-linking may be used if irreversible attachment is desired.

By synthesizing polynucleotide, polypeptide, or specific binding molecule in situ or otherwise attaching it to a substrate at a predetermined, discrete position or to a coded substrate, an interacting polynucleotide, polypeptide, or specific binding molecule can be identified without determining its sequence. For example, a polynucleotide, polypeptide, or specific binding molecule of known sequence can be determined by its position (e.g., rectilinear or polar coordinates) or decoding its signal (e.g., combinatorial tag, electromagnetic radiation) on the substrate. A nucleotide or amino acid sequence will be correlated with each position on or decoded signal of the substrate. A substrate may have a pattern of different polynucleotides, polypeptides, and/or specific binding molecules (e.g., at least 5, 10, 20, 30, 40, 50, 60, 80, 100, 150, 200, 250, 300, 350, 400, 450, 500, 1000, 2000, 3000, 4000, 5000, 7500, 10,000, 50,000, 100,000 or 1,000,000 distinguishable positions) at low or high density (e.g., at least 1,000, 10,000, 100,000 or 1,000,000 distinguishable positions per cm²). The number of molecules that can be differentiated by the signal is only limited by factors such as the scale of the reaction; the number and complexity of combinations; interference with a property of electromagnetic radiation like wavelength, frequency, energy, polarization; etc.

Multiplex analysis may be used to monitor expression of different genes at the same time in parallel. Such multiplex analysis may be performed using different polynucleotides, polypeptides, or specific binding molecules arranged in high density on a substrate. Simultaneous solution methods such as multiprobe ribonuclease protection assay or multiprimer pair amplification associate each transcript with a different length of detected product which is resolved by separation on the basis of molecular weight. Multiplex analysis may include custom-made diagnostics arrays for vascular disorder in AD, and could be compared with the proprietary Socratech data bases.

Changes in gene expression may be manifested in the cell by affecting transcriptional initiation, transcript stability, translation of transcript into protein product, protein stability, or combinations thereof. The gene, transcript, or polypeptide can be assayed by techniques such as in vitro transcription, in vitro translation, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), electrophoretic mobility shift.assay (EMSA), radioimmunoassay (RIA), fluorescent or histochemical staining, microscopy and digital image analysis, and fluorescence activated cell analysis or sorting (FACS).

A reporter or selectable marker gene whose protein product is easily assayed may be used for convenient detection. Reporter genes include, for example, alkaline phosphatase, β-galactosidase (LacZ), chloramphenicol acetyltransferase (CAT), β-glucoronidase (GUS), bacterial/insect/marine invertebrate luciferases (LUC), green and red fluorescent proteins (GFP and RFP, respectively), horseradish peroxidase (HRP), β-lactamase, and derivatives thereof (e.g., blue EBFP, cyan ECFP, yellow-green EYFP, destabilized GFP variants, stabilized GFP variants, or fusion variants sold as LIVING COLORS fluorescent proteins by Clontech). Reporter genes would use cognate substrates that are preferably assayed by a chromogen, fluorescent, or luminescent signal. Alternatively, assay product may be tagged with a heterologous epitope (e.g., FLAG, MYC, SV40 T antigen, glutathione transferase, hexahistidine, maltose binding protein) for which cognate antibodies or affinity resins are available.

A polynucleotide may be ligated to a linker oligonucleotide or conjugated to one member of a specific binding pair (e.g., antibody-digoxygenin/hapten/peptide epitope, biotin-avidin/streptavidin, glutathione S transferase or GST-glutathione, lectin-sugar, maltose binding protein-maltose, polyhistidine-nickel, protein A/G-immunoglobulin). The polynucleotide may be conjugated by ligation of a nucleotide sequence encoding the binding member. A polypeptide may be joined to one member of the specific binding pair by producing the fusion encoded such a ligated or conjugated polynucleotide or, alternatively, by direct chemical linkage to a reactive moiety on the binding member by chemical cross-linking. Such polynucleotides and polypeptides may be used as an affinity reagent to identify, to isolate, and to detect interactions that involve specific binding of a transcript or protein product of the expression vector. Before or after affinity binding of the transcript or protein product, the member attached to the polynucleotide or polypeptide may be bound to its cognate binding member. This can produce a complex in solution or immobilized to a support. A protease recognition site (e.g., for enterokinase, Factor Xa, ICE, secretases, thrombin) may be included between adjoining domains to permit site specific proteolysis that separates those domains and/or inactivates protein activity.

Construction of Expression Vector

An expression vector is a recombinant polynucleotide that is in chemical form either a deoxyribonucleic acid (DNA) and/or a ribonucleic acid (RNA). The physical form of the expression vector may also vary in strandedness (e.g., single-stranded or double-stranded) and topology (e.g., linear or circular). The expression vector is preferably a double-stranded deoxyribonucleic acid (dsDNA) or is converted into a dsDNA after introduction into a cell (e.g., insertion of a retrovirus into a host genome as a provirus). The expression vector may include one or more regions from a mammalian gene expressed in the vascular system, especially endothelial cells (e.g., ICAM-2, tie), or a virus (e.g., adenovirus, adeno-associated virus, cytomegalovirus, fowlpox virus, herpes simplex virus, lentivirus, Moloney leukemia virus, mouse mammary tumor virus, Rous sarcoma virus, SV40 virus, vaccinia virus), as well as regions suitable for genetic manipulation (e.g., selectable marker, linker with multiple recognition sites for restriction endonucleases, promoter for in vitro transcription, primer annealing sites for in vitro replication). The expression vector may be associated with proteins and other nucleic acids in a carrier (e.g., packaged in a viral particle) or condensed with chemicals (e.g., cationic polymers) to target entry into a cell or tissue.

The expression vector further comprises a regulatory region for gene expression (e.g., promoter, enhancer, silencer, splice donor and acceptor sites, polyadenylation signal, cellular localization sequence). Transcription can be regulated by tetracyline or dimerized macrolides. The expression vector may be further comprised of one or more splice donor and acceptor sites within an expressed region; Kozak consensus sequence upstream of an expressed region for initiation of translation; and downstream of an expressed region, multiple stop codons in the three forward reading frames to ensure termination of translation, one or more mRNA degradation signals, a termination of transcription signal, a polyadenylation signal, and a 3′ cleavage signal. For expressed regions that do not contain an intron (e.g., a coding region from a cDNA), a pair of splice donor and acceptor sites may or may not be preferred. It would be useful, however, to include mRNA degradation signal(s) if it is desired to express one or more of the downstream regions only under the inducing condition. An origin of replication may also be included that allows replication of the expression vector integrated in the host genome or as an autonomously replicating episome. Centromere and telomere sequences can also be included for the purposes of chromosomal segregation and protecting chromosomal ends from shortening, respectively. Random or targeted integration into the host genome is more likely to ensure maintenance of the expression vector but episomes could be maintained by selective pressure or, alternatively, may be preferred for those applications in which the expression vector is present only transiently.

An expressed region may be derived from any gene of interest, and be provided in either orientation with respect to the promoter; the expressed region in the antisense orientation will be useful for making cRNA and antisense polynucleotide. The gene may be derived from the host cell or organism, from the same species thereof, or designed de novo; but it is preferably of archael, bacterial, fungal, plant, or animal origin. The gene may have a physiological function of one or more nonexclusive classes: adhesion proteins; cytokines, hormones, and other regulators of cell growth, mitosis, meiosis, apoptosis, senescence, differentiation, or development; soluble or membrane receptors for such factors; adhesion molecules; cell-surface receptors and ligands thereof; cytoskeletal and extracellular matrix proteins; cluster differentiation (CD) antigens, antibody and T-cell antigen receptor chains, histocompatibility antigens, and other factors mediating specific recognition in immunity; chemokines, receptors thereof, and other factors involved in inflammation; enzymes producing lipid mediators of inflammation and regulators thereof; clotting and complement factors; ion channels and pumps; transporters and binding proteins; neurotransmitters, neurotrophic factors, and receptors thereof; cell cycle regulators, oncogenes, and tumor suppressors; other transducers or components of signaling pathways; proteases and inhibitors thereof; catabolic or metabolic enzymes, and regulators thereof. Some genes produce alternative transcripts, encode subunits that are assembled as homopolymers or heteropolymers, or produce propeptides that are activated by protease cleavage. The expressed region may encode a translational fusion; open reading frames of the regions encoding a polypeptide and at least one heterologous domain may be ligated in register. If a reporter or selectable marker is used as the heterologous domain, then expression of the fusion protein may be readily assayed or localized. The heterologous domain may be an affinity or epitope tag.

One or more genes involved in abnormal responses of BEC to aniogenic signaling, aberrant angiogenesis and/or cellular senescence of BEC (e.g., normal or defective) may be expressed or their expression inhibited by the above.

Screening of Candidate Compounds

Another aspect of the invention are chemical or genetic compounds, derivatives thereof, and compositions including same that are effective in treatment of Alzheimer's disease and individuals at risk thereof. The amount that is administered to an individual in need of therapy or prophylaxis, its formulation, and the timing and route of delivery is effective to reduce the number or severity of symptoms, to slow or limit progression of symptoms, to inhibit expression of one or more genes that are transcribed at a higher level in Alzheimer's disease, to activate expression of one or more genes that are transcribed at a lower level in Alzheimer's disease, or any combination thereof. The efficacy of a candidate compound can be determined by comparing its effects on a subset of disease specific genes in a BEC gene expression data base and on altered AD BEC cellular responses (i.e., phenotype). Determination of such amounts, formulations, and timing and route of drug delivery is within the skill of persons conducting in vitro assays, in vivo studies of animal models, and human clinical trials.

A screening method may comprise administering a candidate compound to an organism or incubating a candidate compound with a cell, and then determining whether or not gene expression is modulated. Such modulation may be an increase or decrease in activity that partially or fully compensates for a change that is associated with or may cause Alzheimer's disease. Gene expression may be increased at the level of rate of transcriptional initiation, rate of transcriptional elongation, stability of transcript, translation of transcript, rate of translational initiation, rate of translational elongation, stability of protein, rate of protein folding, proportion of protein in active conformation, functional efficiency of protein (e.g., activation or repression of transcription), or combinations thereof. See, for example, U.S. Pat. Nos. 5,071,773 and 5,262,300. High-throughput screening assays are possible (e.g., by using parallel processing and/or robotics).

The screening method may comprise incubating a candidate compound with a cell containing a reporter construct, the reporter construct comprising transcription regulatory region covalently linked in a cis configuration to a downstream gene encoding an assayable product; and measuring production of the assayable product. A candidate compound which increases production of the assayable product would be identified as an agent which activates gene expression while a candidate compound which decreases production of the assayable product would be identified as an agent which inhibits gene expression. See, for example, U.S. Pat. Nos. 5,849,493 and 5,863,733.

The screening method may comprise measuring in vitro transcription from a reporter construct in the presence or absence of a candidate compound (the reporter construct comprising a transcription regulatory region) and then determining whether transcription is altered by the presence of the candidate compound. In vitro transcription may be assayed using a cell-free extract, partially purified fractions of the cell, purified transcription factors or RNA polymerase, or combinations thereof. See, for example, U.S. Pat. Nos. 5,453,362; 5,534,410; 5,563,036; 5,637,686; 5,708,158; and 5,710,025.

Techniques for measuring transcriptional or translational activity in vivo are known in the art. For example, a nuclear run-on assay may be employed to measure transcription of a reporter gene. Translation of the reporter gene may be measured by determining the activity of the translation product. The activity of a reporter gene can be measured by determining one or more of transcription of polynucleotide product (e.g., RT-PCR of GFP transcripts), translation of polypeptide product (e.g., immunoassay of GFP protein), and enzymatic activity of the reporter protein per se (e.g., fluorescence of GFP or energy transfer thereof).

A compound may be screened for its effect on angiogenesis and/or cellular senescence (e.g., normal or defective) in accordance with the above.

Genetic Compounds for Treatment

Gene activation may be achieved by inducing an expression vector with a downstream region related to a gene which is down regulated in Alzheimer's disease (e.g., the full-length coding region or functional portions of the gene; hypermorphic mutants, homologs, orthologs, or paralogs thereof) or unrelated to the gene that acts to relieve suppression of gene activation (e.g., at least partially inhibiting expression of a negative regulator of the gene). Over expression of transcription or translation, as well as over expressing protein function, is a more direct approach to gene activation. Alternatively, the downstream expressed region may direct homologous recombination into a locus in the genome and thereby replace or supplement an endogenous transcriptional regulatory region of the gene with an expression cassette.

An expression vector may be introduced into a host mammalian cell or tissue, or nonhuman mammal by a transfection or transgenesis technique using, for example, one or more chemicals (e.g., calcium phosphate, DEAE-dextran, lipids, polymers), biolistics, electroporation, naked DNA technology, microinjection, or viral infection. Osmotic shock or surgical procedures may also be used for transfer across the BBB to stimulate transport of vectors into BEC at the abluminal BBB site or at the luminal site. The introduced expression vector may integrate into the host genome of the mammalian cell or nonhuman mammal, or be maintained as an episome. Many neutral and charged lipids, sterols, and other phospholipids to make lipid carriers are known. For example, neutral lipids are dioleoyl phosphatidylcholine (DOPC) and dioleoyl phosphatidyl ethanolamine (DOPE); an anionic lipid is dioleoyl phosphatidyl serine (DOPS); cationic lipids are dioleoyl trimethyl ammonium propane (DOTAP), dioctadecyidiamidoglycyl spermine (DOGS), dioleoyl trimethyl ammonium (DOTMA), and 1,3-di-oleoyloxy-2-(6-carboxyspermyl)-propylamide tetraacetate (DOSPER). Dipalmitoyl phosphatidylcholine (DPPC) can be incorporated to improve the efficacy and/or stability of delivery. Proprietary lipid formulations include: FUGENE 6, LIPOFECTAMINE, LIPOFECTIN, DMRIE-C, TRANSFECTAM, CELLFECTIN, PFX-1, PFX-2, PFX-3, PFX4, PFX-5, PFX-6, PFX-7, PFX-8, TRANSFAST, TFX-10, TFX-20, TFX-50, and LIPOTAXI. The polymer may be cationic dendrimers, polyamides, polyamidoamines, polyethylene or polypropylene glycols (PEG), polyethylenimines (PEI), polylysines, or combinations thereof; alternatively, polymeric materials can be formed into nanoparticles or microparticles. In naked DNA technology, the expression vector (usually as a plasmid) is delivered to a cell or tissue, where it may or may not become integrated into the host genome, without using chemical transfecting agents (e.g., lipids, polymers) to condense the expression vector prior to its introduction into the cell or tissue.

A mammalian cell may be transfected with an expression vector; also provided are transgenic nonhuman mammals made by inserting a construct into the nucleus at a random or targeted location, or as an episome. In the previously discussed alternative, a homologous region from a gene can be used to direct integration to a particular genetic locus in the host genome and thereby regulate expression of the gene at that locus or ectopic copies of the gene may be inserted. For example, a knock-out mutation would eliminate gene function and a knock-in mutation would replace the host sequence with a nucleotide sequence of the mutant construct (e.g., neomorphic, hypomorphic, hypermorphic). Polypeptide may be produced in vitro by culturing transfected cells, in vivo by transgenesis, or ex vivo by introducing an expression vector into allogeneic, autologous, histocompatible, or xenogeneic cells and then transplanting the transfected cells (e.g., totipotent or pluripotent stem cell) into a host organism. Special harvesting and culturing protocols will be needed for transfection and subsequent transplantation of host stem cells into a host mammal. Immunosuppression of the host mammal post-transplant or encapsulation of the host cells may be necessary to prevent rejection.

The expression vector may be used to replace function of a gene that is down regulated or totally defective, supplement function of a partially defective gene, or compete with activity of the gene. Thus, the cognate gene activity of the host may be neomorphic, hypomorphic, hypermorphic, or normal. Replacement or supplementation of function can be accomplished by the methods discussed above, and transfected mammalian cells or transgenic nonhuman mammals may be selected for high or low expression (e.g., assessing amount of transcribed or translated produce, or physiological function of either product) of the downstream region. But competition between the expressed downstream region and a neomorphic, hypermorphic, or normal gene may be more difficult to achieve unless the encoded polypeptides are multiple subunits that form into a polymeric protein complex. Alternatively, a negative regulator or a single-chain antibody that inhibits function intracellularly may be encoded by the downstream region of the expression vector. Therefore, at least partial inhibition of genes that are up regulated in MBEC of Alzheimer's disease may use antisense, ribozyme, RNAi, or triple helix technology in which the expression vector contains a downstream region corresponding to the unmodified antisense molecule, ribozyme, siRNA duplex, or triple helix molecule, respectively.

Antisense polynucleotides were initially.believed to directly block translation by hybridizing to mRNA but may involve degradation of such transcripts of a gene. The antisense molecule may be recombinantly made using at least one functional portion of a gene in the antisense orientation as a downstream expressed region in an expression vector. Chemically modified bases or linkages may be used to stabilize the antisense polynucleotide by reducing degradation or increasing half-life in the body (e.g., methyl phosphonates, phosphorothioate, peptide nucleic acids). The sequence of the antisense molecule may be complementary to the translation initiation site (e.g., between −10 and +10 of the target's nucleotide sequence).

Ribozymes catalyze specific cleavage of an RNA transcript or genome. The mechanism of action involves sequence-specific hybridization to complementary cellular or viral RNA, followed by endonucleolytic cleavage. It may or may not be dependent on ribonuclease H activity. The ribozyme includes one or more sequences complementary to the subject RNA as well as catalytic sequences responsible for RNA cleavage (e.g., hammerhead, hairpin, axehead motifs). For example, potential ribozyme cleavage sites within a subject RNA are initially identified by scanning the subject RNA for ribozyme cleavage sites which include the following trinucleotide sequences: GUA, GUU and GUC. Once identified, an oligonucleotide of between about 15 and about 20 ribonucleotides corresponding to the region of the subject RNA containing the cleavage site can be evaluated for predicted structural features, such as secondary structure, that can render candidate oligonucleotide sequences unsuitable. The suitability of candidate sequences can then be evaluated by their ability to hybridize and cleave target RNA.

siRNA refers to double-stranded RNA of at least 20-25 basepairs which mediates RNA interference (RNAi). Duplex siRNA corresponding to a target RNA may be formed by separate transcription of the strands, coupled transcription from a pair of promoters with opposing polarities, or annealing of a single RNA strand having an at least partially self-complementary sequence. Alternatively, duplexed oligoribonucleotides of at least about 21 to about 23 basepairs may be chemically synthesized (e.g., a duplex of 21 ribonucleotides with 3′ overhangs of two ribonucleotides) with some substitutions by modified bases being tolerated. Mismatches in the center of the siRNA sequence, however, abolishes interference. The region targeted by RNA interference should be transcribed, preferably as a coding region of the gene. Interference appears to be dependent on cellular factors (e.g., ribonuclease III) that cleave target RNA at sites 21 to 23 bases apart; the position of the cleavage site appears to be defined by the 5′ end of the guide siRNA rather than its 3′ end. Priming by a small amount of siRNA may trigger interference after amplification by an RNA-dependent RNA polymerase.

Molecules used in triplex helix formation for inhibiting expression of a gene that is up regulated should be single-stranded and composed of deoxyribonucleotides. The base composition of these oligonucleotides must be designed to promote triple helix formation by Hoogsteen base pairing rules, which generally require sizeable stretches of either purines or pyrimidines to be present on one strand of the duplex. Nucleotide sequences can be pyrimidine-based and result in TAT and CGC triplets across the three associated strands. The pyrimidine-rich molecules provide base complementarity to a purine-rich region of a single strand of the duplex in a parallel orientation to that strand. In addition, triple helix forming molecules can be chosen that are purine-rich (e.g., containing a stretch of guanines). These molecules may form a triple helix with a DNA duplex that is rich in GC pairs, in which the majority of the purines are located on a single strand of the targeted duplex, resulting in GGC triplets across the three strands in the triplex.

Antibody specific for a gene product increased in Alzheimer's disease can be used for inhibition or detection. Polyclonal or monoclonal antibodies may be prepared by immunizing animals (e.g., chicken, hamster, mouse, rat, rabbit, goat, horse) with antigen, and optionally affinity purified against the same or a related antigen. Antibody fragments may be prepared by proteolytic cleavage or genetic engineering; humanized antibody and single-chain antibody may be prepared by transplanting sequences from the antigen binding domains of antibodies to framework molecules. In general, other specific binding molecules may be prepared by screening a combinatorial library for a member which specifically binds antigen (e.g., phage display library). Antigen may be a full-length protein encoded by the gene or fragment(s) thereof. See, for example, U.S. Pat. Nos. 5,403,484; 5,723,286; 5,733,743; 5,747,334; and 5,871,974.

Genes involved in abnormal responses to angiogenic signaling, aberrant brain capillary morphogenesis and BEC differentiation and/or cellular senescence (e.g., normal or defective) may be expressed or their expression inhibited by the above.

Formulation of Compositions

Compounds of the invention or derivatives thereof may be used as a medicament or used to formulate a pharmaceutical composition with one or more of the utilities disclosed herein. They may be administered in vitro to cells in culture, in vivo to cells in the body, or ex vivo to cells outside of an individual that may later be returned to the body of the same individual or another. Such cells may be disaggregated or provided as solid tissue. Examples of drugs that prevent aberrant AD BEC-mediated or SIPS BEC-mediated brain capillary tube formation during in vitro assays (i.e., reverse the dysfunctional vascular phenotype) are presented below.

Compounds or derivatives thereof may be used to produce a medicament or other pharmaceutical compositions. Use of.compositions which further comprise a pharmaceutically acceptable carrier and compositions which further comprise components useful for delivering the composition to an individual are known in the art. Addition of such carriers and other components to the composition of the invention is well within the level of skill in this art.

Pharmaceutical compositions may be administered as a formulation adapted for passage through the blood-brain barrier or direct contact with the endothelium. Alternatively, pharmaceutical compositions may be added to the culture medium. In addition to the active compound, such compositions may contain pharmaceutically-acceptable carriers and other ingredients known to facilitate administration and/or enhance uptake (e.g., saline, dimethyl sulfoxide, lipid, polymer, affinity-based cell specific-targeting systems). The composition may be incorporated in a gel, sponge, or other permeable matrix (e.g., formed as pellets or a disk) and placed in proximity to the endothelium for sustained, local release. The composition may be administered in a single dose or in multiple doses which are administered at different times.

Pharmaceutical compositions may be administered by any known route. By way of example, the composition may be administered by a mucosal, pulmonary, topical, or other localized or systemic route (e.g., enteral and parenteral). The term “parenteral” includes subcutaneous, intradermal, intramuscular, intravenous, intraarterial, intrathecal, and other injection or infusion techniques, without limitation.

Suitable choices in amounts and timing of doses, formulation, and routes of administration can be made with the goals of achieving a favorable response in the individual with Alzheimer's disease or at risk thereof (i.e., efficacy), and avoiding undue toxicity or other harm thereto (i.e., safety). Therefore, “effective” refers to such choices that involve routine manipulation of conditions to achieve a desired effect.

A bolus of the formulation administered to an individual over a short time once a day is a convenient dosing schedule. Alternatively, the effective daily dose may be divided into multiple doses for purposes of administration, for example, two to twelve doses per day. Dosage levels of active ingredients in a pharmaceutical composition can also be varied so as to achieve a transient or sustained concentration of the compound or derivative thereof in an individual, especially in and around vascular endothelium of the brain, and to result in the desired therapeutic response or protection. But it is also within the skill of the art to start doses at levels lower than required to achieve the desired therapeutic effect and to gradually increase the dosage until the desired effect is achieved.

The amount of compound administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration, and the like. For systemic administration, passage of the compound or its metabolite through the blood-brain barrier is important. It will also be understood that the specific dose level to be achieved for any particular individual may depend on a variety of factors, including age, gender, health, medical history, weight, combination with one or more other drugs, and severity of disease.

The term “treatment” of Alzheimer's disease refers to, inter alia, reducing or alleviating one or more symptoms in an individual, preventing one or more symptoms from worsening or progressing, promoting recovery or improving prognosis, and/or preventing disease in an individual who is free therefrom as well as slowing or reducing progression of existing disease. For a given individual, improvement in a symptom, its worsening, regression, or progression may be determined by an objective or subjective measure. Efficacy of treatment may be measured as an improvement in morbidity or mortality (e.g., lengthening of survival curve for a selected population). Prophylactic methods (e.g., preventing or reducing the incidence of relapse) are also considered treatment. Treatment may also involve combination with other existing modes of treatment (e.g., ARICEPT or donepezil, COGNEX or tacrine, EXELON or rivastigmine, REMINYL or galantamine, antiamyloid vaccine, Aβ-lowering therapies, mental exercise or stimulation; see for review Zlokovic, Adv. Drug Deliv. Rev. 54:1533-1660, 2002). Thus, combination treatment with one or more other drugs and one or more other medical procedures may be practiced.

Similarly, diagnosis according to the invention may be practiced with other diagnostic procedures. For example, endothelium of the vascular system, brain, or spinal cord (e.g., blood or leptomeningeal vessels) may be assayed for a change in gene expression profiles using disease-specific molecular diagnostics kits (e.g., custom made arrays, multiplex QPCR, multiplex proteomic arrays). In addition, a noninvasive diagnostic procedure (e.g., CAT, MRI, SPECT, or PET) may be used in combination to improve the accuracy and/or sensitivity of diagnosis. Early and reliable diagnosis is especially useful to for treatments that are only effective for mild to moderate Alzheimer's disease or only delay its progression.

The amount which is administered to an individual is preferably an amount that does not induce toxic effects which outweigh the advantages which result from its administration. Further objectives are to reduce in number, diminish in severity, and/or otherwise relieve suffering from the symptoms of the disease in the individual in comparison to recognized standards of care. The invention may also be effective against neurodegenerative disorders or cognitive impairment in general: for example, dementia, depression, confusion, Creutzfeldt-Jakob or mad cow disease, Huntington's disease, loss of motor coordination, multiple sclerosis, Parkinson's disease, Pick disease and other brain storage disorders (e.g., amyloidosis, gangliosidosis, lipid storage disorders, mucopolysaccharidosis), stroke, syncope, and vascular dementia. Thus, treatment may be directed at an individual who is affected or unaffected by the neurodegenerative disease; it may improve cognitive function. The efficacy of treatment may be determined by monitoring cerebral blood flow (CBF) and/or blood-brain barrier (BBB) function.

Production of compounds according to present regulations will be regulated for good laboratory practices (GLP) and good manufacturing practices (GMP) by governmental agencies (e.g., U.S. Food and Drug Administration). This requires accurate and complete record keeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

The following examples substantiate the claims, inter alia, that there is dysregulation of vascular function in Alzheimer's disease: e.g., abnormal responses to angiogenic signaling, apoptosis, aniokis, and/or mitotic catastrophe during brain capillary morphogenesis, aberrant capillary formation throughout all stages (stage I—vaculolization, stage II—tube formation, stage III—network formation, stage IV—remodeling), cellular senescence, presence of nonfunctional capillaries, and loss of BBB and CBF regulatory functions, which are all related to changes in expression of disease-specific genes. This can be used as a prognostic indication for diagnosis and treatment. But they are merely illustrative of the invention, and are not intended to restrict or otherwise limit its practice.

### EXAMPLES

Human Subjects

Microvascular brain endothelial cells (BEC) are representative of the site of the BBB. They were cultured from human brain tissue (Brodmann's areas 9 and 10) obtained at autopsy with the postmortem interval (PMI) typically between 3 hr and 6 hr, or from biopsy during brain surgery for epilepsy or brain trauma. The groups of patients and controls used for different studies are given in Tables 1-8. Each Table contains provides information on age, gender, PMI, cause of death, the presence of vascular risk factors, angiopathy, Braak stage, CERAD stage, and CDR (cognitive dementia rate) score for each studied individual. Total RNA was isolated from primary cultures of BEC at passages 2-4 (P2 to P4).

In our first gene expression analysis using Affymetrix U95A chips (12,500 genes), we compared six AD patients (Table 1) with six age-matched controls (Table 2), corrected for normal aging process with-five young controls (Table 3). The characteristics of studied AD cases vs. age-matched controls were: age, 72.5±3.45 vs. 72.2±6.01 (mean±s.e., years), gender ratio female/male (F=0 and M=1) of 0.43 vs. 0.50, PMI of 3.80±0.41 vs. 4.51±0.75 (mean±s.e., hr), cause of death in both groups were similar (e.g., cardiac arrest, respiratory failure), incidence of vascular risk factors 4/6 vs. 4/6 was similar, the presence of amyloid angiopathy were 6/6 vs. 2/6, Braak stage V-VI vs. 0-0I, CERAD F/M ratio in AD of 0.5 vs. F/M ratio in controls of 0.5, CDR 3.83±0.29 (AD cases) vs. 0.08±0.03 (age-matched controls). Characteristics of young controls were: age of 23.4±3.80 (mean±s.e., years), F/M ratio of 0.6, PMI of 4.46±0.62 (mean±s.e., hours), cause of death was trauma, no vascular risk factors; Braak and CERAD were zero in all cases (not shown in Table 3), and family history did not reveal cognitive problems (CDR was not determined).

In our second gene expression analysis using Affymetrix U133A and U133B chips (45,000 genes), 11 AD patients (Table 4) were compared with five age-matched controls (Table 5), corrected for normal aging process by five middle age controls (Table 6), and five young controls (Table 7). The characteristics of AD cases vs. age-matched controls were: age, 80.6±3.65 vs. 82.0±3.87 (mean±s.e., years), gender ratio female/male (F=0 and M=1) of 0.45 vs. 0.40, PMI of 3.64±0.26 vs. 3.53±0.96 (mean±s.e., hours), cause of death in both groups were similar (e.g., cardiac arrest, respiratory failure), incidence of vascular risk factors was similar 8/11 vs. 4/5, the presence of amyloid angiopathy was 9/11 vs. 3/5, Braak stage V-VI for AD vs. 0-I-I-II for age-matched, CERAD F/M ratio for AD of 0.72 vs. F/M ratio for age-matched of 0.4, CDR 3.83±0.29 (AD) vs. 0.08±0.03 (controls). Characteristics of middle age controls were: age of 59.4±1.20 (mean±s.e., years), F/M ratio of 0.4, PMI of 4.75±0.22 (mean±s.e., hours), cause of death cardiac arrest, respiratory, incidence of vascular risk factors 2/5, angiopathy 0/5, Braak 0, CERAD 0, CDR 0. Characteristics of young controls were: age of 27.6±4.67 (mean±s.e., years), F/M ratio 0.4, PMI 4.46±0.62 (mean±s.e., hours), cause of death was trauma, no vascular risk factors; Braak and CERAD were zero in all cases (not shown in Table 7), and family history did not reveal cognitive problems (CDR was not determined).

Table 8 illustrate cases used in the senescence study. The characteristics of studied eight AD cases vs. five age-matched controls were: age, 78.3±3.71 vs. 72.0±7.39 (mean±s.e., years), gender ratio female/male (F=0 and M=1) 0.5 vs. 0.6, PMI of 3.85±0.93 vs. 4.50±0.97 (mean±s.e., hours), cause of death in both groups were similar, i.e., cardiac or respiratory arrest (not shown in Table 8), incidence of vascular risk factors was comparable in both groups between 60 and 70% of cases had a vascular risk factor (not shown in Table 8), the presence of amyloid angiopathy was 6/8 vs. 0/5, Braak stage V-VI for AD vs. 0-0-I for controls, CERAD F/M ratio for AD of 0.62 vs. 0 for controls, CDR in AD close to 4 in controls 0 (not shown in Table 8). Young controls were: age of 19.3.6±3.53 (mean±s.e., years), F/M ratio of 0.7, PMI (biopsy cases), no vascular risk factors; no angiopathy, CERAD zero, CDR zero (not shown in Table 8).

Neuropathological Analysis

Tissue blocks (1 cm³) from autopsy cases were fixed in 10% neutral-buffered formalin, pH 7.3 (Sigma), and embedded in paraffin or snap-frozen in liquid nitrogen-chilled isopentane. The tissue samples were obtained from the superior and middle frontal gyrus (Brodmann's areas 9 and 10). Tissue sections were stained with either hematoxylin and eosin (H&E) stain or thioflavin S by a modified Bielschowsky silver impregnation method (Gallyas stain). Thioflavin S stained sections were viewed through a Zeiss fluorescence microscope equipped with a narrow band, blue/violet filter from 400 nm to 455 nm. Two independent observers performed the examination. Diagnosis of Alzheimer's disease was made according to a modified CERAD (Consortium to Establish a Registry for Alzheimer's Disease) protocol (see Hyman and Trojanowski, J. Neuropathol Exp. Neurol. 56:1095-1097,1997). In most cases, Braak analysis was performed in parallel.

Isolation and Culture of Human BEC.

BEC were isolated postmortem as reported (Mackic et al., J. Clin. Invest. 102:734-743,1998). Briefly, brain tissue was cut into small pieces, and then mechanically dissociated using a loose-fitting cell homogenizer in RPMI 1640 with 2% fetal calf serum (FCS) and penicillin/streptomycin. The homogenate was then fractionated over 15% dextran by centrifugation at 10,000 g for 10 min to obtain a brain microvessel pellet. Microvessels were further digested with 1 mg/ml of collagenase/dispase and 5 μl/ml of DNase in FCS-enriched medium for 1 hr at 37° C. Subsequently the cell suspension was centrifuged at 1000 g for 5 min, and the cell pellet was plated on fibronectin-coated flasks in RPMI 1640 with 10% FCS, 10% NuSerum, endothelial cell growth factors, nonessential amino acids, vitamins, and penicillin/streptomycin (Mackic et al., J. Clin. Invest. 102:734-743, 1998).

Characterization of BEC

The P0 primary cultures were grown to confluence, and sorted based on LDL binding using the Dil-Ac-LDL method following the manufacturer's instructions (Biomedical Technology). Briefly, cells were incubated with Dil-Ac-LDL ligand for 4 hr at 37° C., trypsinized, and then separated by fluorescence activated 25 cell sorting (FACS). Labeled and unlabeled human umbilical vein endothelial cells (HUVEC) were used to set gating limits as positive and negative controls, respectively. Unlabeled MBEC were used to control for possible background staining or differences based on cell size. Positively sorted cells were plated on fibronectin- or collagen-coated flasks in the medium described above. Cultures were grown in 5% CO₂ and split 1:3 at confluency with collagenase/dispase (Mackic et al., J.

Clin. Invest. 102:734-743,1998). Cells were characterized on cytospins (cells centrifuged onto slides) with a panel of cell-specific antibodies including antibodies against Factor VIII or CD105 (endothelium), CD11b (monocyte/microglia), glial fibrillar acidic protein (astrocytes), a-actin (vascular smooth muscle), and neurofilament-α (neurons). Cells were greater than 98% positive for Factor VIII and CD105, but negative for the other markers confirming their endothelial origin.

Immunocytochemical Analysis

Air-dried cryostat sections (10 μm) of the frontal cortex adjacent to the BEC isolation site and cytospins of subconfluent BEC were used for immunocytochemical analysis. For single staining, after incubation with primary antibody, preparations were treated with biotinylated secondary IgG and incubated with avidin-biotin-HRP (Vector Laboratories). Binding was detected with an SG peroxidase detection kit (blue/gray; Vector Laboratories). For double labeling, after incubation with the second primary antibody, sections were treated with biotinylated IgG, and detected with NovaRED (Vector Laboratories). Imaging was accomplished using an Axiophot II microscope (Carl Zeiss) equipped with SPOT digital camera. Some examples of antibodies used for immunocytochemical analysis include Aβ₄₀, rabbit anti-human,1:1,000 (1 mg/ml, Chemicon Intl.); Aβ₄₂, rabbit anti-human, 1:1,000 (1 mg/ml); gax, rabbit polyclonal against C-terminal region of the rat gax protein (amino acids SDHSSEHAHL), 1:500 (7 mg/ml, provided by Dr Kenneth Walsh); integrins avβ3 and avβ5 (Chemicon Intl.); the mouse monoclonal antibody to the heavy chain of human LRP-1 designated 8G1, which is specific for human LRP-1 and recognizes an epitope on the 515 kDa subunit, 1:300 (1.5 mg/ml); cyclin B2, goat anti-human polyclonal, 1:100 (0.2 mg/ml, Santa Cruz Biotechnology); CD105 (clone SMG), mouse anti-human, 1:100 (0.1 mg/ml, Serotec); Von Willebrand Factor, rabbit anti-human monoclonal, 1:200 (5.6 mg/ml, DAKO); GFAP, mouse anti-bovine polyclonal, 1:500 (11.7 mg/ml, DAKO); CD11 b, mouse anti-human monoclonal, 1:500 (4.2 mg/ml, DAKO); α-actin, mouse anti-human monoclonal, 1:100 (0.2 mg/ml, Oncogene); plectin, goat anti-human polyclonal, 1:100 (0.2 mg/ml, Santa Cruz Biotechnology); AFX-1, rabbit anti-human polyclonal, 1:1,000 (0.8 mg/ml, Sigma); tissue transglutaminase TG2, rabbit anti-human polyclonal, 1:200 (0.2 mg/ml, Calbiochem); E2F transcription factor, rabbit anti-human polyclonal, 1:1,000 (0.2 mg/ml, Santa Cruz Biotechnology); MMP1, goat anti-human polyclonal, 1:1,000 (0.2 mg/ml, Santa Cruz Biotechnology), ankyrin G, mouse anti-human, 1:1,000 (0.2 mg/ml, Santa Cruz Biotechnology); p53, mouse anti-human, 1:100 (0.4 mg/ml, DAKO); human active caspase-3, 1:250 (1 mg/ml; Promega); and p16, mouse anti-human, 1:50 (0.03 mg/ml, BD Pharmingen).

Capillary Morphogenesis Assays

The 3-D system has been described in detail by Davis et al. (J. Cell Sci. 114:917-930, 2001). Here, the system was used to assay the responsiveness of AD BEC and SIPS BEC to angiogenic stimulation. Briefly, 10⁶ MBEC/ml were suspended within 3-D collagen matrices at 25 μl per well in the serum-free culture medium 199 containing VEGF (40 ng/ml) and bFGF (40 ng/ml) in 5% CO₂ at 37° C. The cells were fixed with 3% glutaraldehyde in phosphate buffered saline. The sections were stained with hematoxylin/eosin and Hoechst 33342. The formation of intracellular vacuoles (stage I), tubules (stage II), and multicellular tubes and networks (stage III) were determined at 4 hr to 16 hr and 24 hr, respectively. The number of apoptotic cells were determined between 4 hr and 24 hr using double TUNEL/Hoechst staining. At least 200 cells were evaluated from an individual well. Light output (lumens) was quantified at 200 X magnification by counting four fields derived from triplicate wells.

In the 2-D system, collagen matrigels of matrix composition containing a supplement of growth factors were used according to manufacturer's instructions (BD Biosciences, Bedford, Mass.). In the 2-D matrix system, 2×10⁴ cells/well were plated.

Drugs Used in Angiogenesis in Vitro Assays

ZVAD-fmk (50 μM, Sigma), p38 MAPK antagonists including SB SB202190 (SB, 10 μM), and plasma-derived activated protein C (APC, prepared by Dr. J. H. Griffin's laboratory, 5-100 nM) were used.

RNA Isolation from MBEC

About 5×10⁵ MBEC were plated in a 100 mm tissue culture dish. MBEC were cultured for 3 to 5 days until the monolayer was subconfluent (about 80%). Total RNA was isolated using TRIZOL reagent (Life Technologies) according to the manufacturer's instructions: cells were homogenized in a monophasic solution comprised of phenol and guanidine isothiocyanate, add chloroform and separate phases, differentially precipitate RNA, and wash and solubilize RNA (U.S. Pat. No. 5,346,994). Total RNA was visualized by gel electrophoresis and analyzed by spectrophotometry to assess the purity and integrity of the preparation.

Preparation of Labeled Target

Total RNA (10 μg) from each sample was used to generate high fidelity cDNA, which was modified at the 3′ end to contain an initiation site for T7 RNA polymerase following the manufacturer's instructions (SUPERCHOICE kit, Life Technologies). Upon completion of cDNA synthesis, 1 μg of product was used in an in vitro transcription (IVT) reaction that contained biotinylated UTP and CTP which were labeled for detection following hybridization to the array following the manufacturer's instructions (ENZO). Full-length IVT product (20 μg) was subsequently fragmented in 200 mM Tris-actetate (pH 8.1), 500 mM KOAc, and 150 mM MgOAc at 94° C. for 35 min. Following fragmentation, all components generated throughout the processing procedure (cDNA, full-length cRNA, and fragmented cRNA) were analyzed by gel electrophoresis to assess the appropriate size distribution prior to array hybridization.

High Density Oligonucleotide Array Hybridization

Samples were subjected to gene expression analysis with the Affymetrix U95A chip (12,500 genes) or Affymetrix U133A and U133B chips (45,000 genes). All procedures have been performed according to the manufacturer's instructions. The detailed protocol for sample processing of Affymetrix microarrays and documentation of the sensitivity and quantitative aspects of the method can be found in the Affymetrix manual.

Data Analysis and Comparative Results

Results with selected genes are discussed below. Although GENECHIP technology was used here, similar results are expected if another array technology was used such as spotted arrays (Affymetrix) or printed arrays (Rosetta). Moreover, differential display (U.S. Pat. No. 5,665,547); serial analysis of gene expression (U.S. Pat. No. 5,866,330, Genyzme); bead arrays analyzed by fiber optics (WO 98/50782, lllumina) or sorting (U.S. Pat. No. 6,265,163, Lynx) are expected to arrive at similar results. Similarly, biosensors to detect protein (U.S. Pat. No. 6,329,209) or a cell (U.S. Pat. No. 6,210,910) can be used for gene expression profiling.

Statistical analysis was performed using Bayesian correction for Affymetrix data (Baldi and Long, Bioinformatics 17:509-519, 2001; Long et al., J. Biol. Chem. 276:19937-19944, 2001). The present analysis used the following criteria: 2-fold ratio, Bayesian adjustment of a signal at 500 (expression), and 0.05 Bayesian p-log.

Quantitative PCR

The same cDNA was used for microarray hybridization and QRT-PCR analysis for a subset of genes. mRNA quantitation was performed using Taq-Man™ chemistry with fluorescently tagged oligonucleotide probes. Fluorescent intensity was detected by the Perkin-Elmer Applied Biosystem Sequence Detector 7700. Data were analyzed using Perkin-Elmer Sequence Detector Software version 1.6.3. Comparative analysis was performed using the delta-delta Ct approach as described by Applied Biosystems.

Western Blot Analysis

Cell lysates were prepared from subconfluent BEC cultures, BEC at different stages of capillary morphogenesis or BEC exposed to SIPS (see below H₂O₂ model of SIPS). Antibodies for Western blot analysis included caspase 3, rabbit anti-human polyclonal, 1:100 (0.5 mg/ml, BD PharMingen); p53, mouse anti-human monoclonal, 1:200 (0.1 mg/ml, Oncogene); p16, mouse anti-human monoclonal, 1:250 (0.5 mg/ml, BD ParMingen); p21, mouse anti-human, 1:200 (0.4 mg/ml, Oncogene); Rb (Santa Cruz), p27 (Santa Cruz), fibronectin (Santa Cruz), PAI-1 (Santa Cruz) or phosphorylated p38 MAPK; gax, rabbit polyclonal against C-terminal region of the rat gax protein (amino acids SDHSSEHAHL), 1:500 (7 mg/ml, provided by Dr Kenneth Walsh); and β-actin, goat anti-human polyclonal, 1:2,500 (0.2 mg/ml, Santa Cruz Biotechnology). The secondary antibody was HRP-conjugated and peroxidase activity was detected with enhanced chemiluminescence detection kit (ECL, Pierce). The relative abundance of the primary antigen was determined by scanning densitometry using β-actin as an internal control.

FACS Analyses of Integrins and Annexin V

FACS analysis of integrins avp3 or avp5 was performed using mouse anti-human avp3 or avp5 antibodies (Chemicon Intl.), respectively, and FITC-goat anti-mouse secondary antibody.

Blood Vessel Quantification and Immunocytochemical Analysis

To determine the size and number of vessels in the gray matter of frontal cortex (areas 9 and 10), tissue sections were stained with anti-CD105 (i.e., endoglin), which labels abluminal site of brain endothelium and anti-CD31 (i.e., PECAM) which labels luminal side of endothelium. Adjacent sections were assessed for amyloid burden with thioflavin S and Gallyas histochemistry, and semiquantified immunohistochemically with anti-Aβ₁₋₄₀ and anti-Aβ₁₋₄₂ staining of plaques and vascular amyloid. Vessels were imaged from four randomly selected fields of cortex (200 μm² each) using an AXIOPHOT microscope (Zeiss) equipped with a SPOT digital camera and PHOTOSHOP software ver. 5.5 (Adobe). Quantification of vessels was based on external endothelial cross-sectional diameters using IMAGEPRO software. Vessels were segregated by size as follows: 6-10 μm (capillaries), 10-30 μm (precapillaries and arterioles), or greater than 30 μm (small arteries).

Senescence-Associated-β-Galactosidase (SA-β-gal)

The proportion of brain endothelial cell positive for SA-β-gal activity was determined as described by Dimri et al. (Proc. Natl. Acad. Sci. USA 92:9363-9367, 1995). Subconfluent cultures were fixed with 2% formaldehyde and 0.2% glutaraldehyde. The presence of SA-β-gal activity was determined by incubation with 1 mg/ml solution of 5-bromo-4-chloro-3-indolyl β-D-galatopyranosoide in 40 mM Na citric acid,. 5 mM K₃FeCN6, 5 mM K₄FeCN₆, 150 mM NaCl, 2 mM MgCl₂ diluted in phosphate-buffered saline (pH 6). The cells were rinsed twice with phosphate buffer saline and washed with methanol.

Cell Cycle Analysis

DNA content was determined with bromodeoxyuridine (BrdU) incorporation and propidium iodide staining as described (Giaretti et al., Exp. Cell Res. 182:290-295, 1989). The brain endothelial cell cells were stained with 50 μg/mL propidium iodide and the S-phase cells were labeled with 10 μg/mL BrdU for 1 hr and washed with serum free medium. DNA content profiles for BrdU-positive cells are shown. The analysis was based on 10,000 cells counted.

TUNEL (Terminal Deoxynucleotidyl Transferase-Mediated in situ end Labeling) Assay

Staining with APO-BRDU kit was performed according to manufacturer's instructions (Phoenix Flow Systems).

Nerve Growth Factor (NGF) ELISA

The NGF ELISA (enzyme-linked immunosorbent assay) reagents were from Promega. The NGF levels were determined in brain endothelial cell cell culture supernatants.

Co-Culture of Endothelial and Neuronal Cells

Endothelial cells were plated on collagen-coated membranes (0.4 μm pores) in the upper chamber of TRANSWELL inserts (Costar) in a dish and cultured in 5% CO₂at 37° C. (Mackic et al., J. Clin. Invest. 102:734-743, 1998). For co-cultures, TRANSWELL inserts containing endothelial cells were rinsed with B27/neurobasal medium and transferred to the plate containing one-day old primary rat hippocampal neurons in the lower chamber. After two days of co-culturing, neurons were fixed in 4% paraformaldehyde and the length of neurites determined using IMAGE-PRO PLUS software (Media Cybernetics).

H₂O₂ Stress-Induced Premature Senescence (SIPS)

Subconfluent BEC cultures (34 days after subculture) were treated with H₂O₂ by adding it to the culture medium for 2 hr. To induce senescence, sublethal doses of H₂O₂ were determined and selected. After treatment, cells were washed with PBS (37° C.) before harvesting, subculturing or incubating with a fresh medium or 3-D collagen gels. H₂O₂-treated cells were subcultured and analyzed after 1, 2, 3, 4, 5, 6, 24, 48, 72, 96 and 120 hr for expression of different proteins involved in the cell cycle regulation and apoptosis. Cells in 60 mm dishes were lysed in SDS sample buffer directly. For Western blot analysis, proteins were separated by SDS-PAGE and transferred to PVDF membrane followed by incubation with different primary antibodies (see antibodies for.Western blot analysis). SIPS BEC were also prepared for microarray analysis on U95A chips as described above.

Differences in Cell Biology

Preliminary findings have been reported in four abstracts: Chow N, Li F, Brooks A, Zidovetzki R, Hofman F, Zlokovic BV, “Senescence of Cerebral Endothelium in Alzheimer's Disease” Soc. Neurosci. Abstract No. 420.9 (2002); Hofman F M, Chow N, Kanagala S, Zidovetzki R, Zlokovic B V, “Down Regulation of Gax in Cerebral Endothelium in Alzheimer's Disease” Soc. Neurosci. Abstract No. 328.2 (2002); Kanagala S, Li F, Chow N, Brooks A, Paxhia A, Armstrong D, Zidovetzki R, Hofman F, Zlokovic B V, “Defective Angiogenesis in Alzheimer's Disease” Soc. Neurosci. Abstract No. 328.2 (2002); and Chow N, Li F, Brooks A, Zidovetzki R, Hofman F, Zlokovic B V, “Cellular senescence of vascular brain endothelium in Alzheimer's disease” Cold Spring Harbor Meeting on Molecular Genetics and Aging Conference (2002). Preliminary findings will be reported in three abstracts: Hofman F M, Li F, Chow N, Cheng T, Penn L, Griffin J H, Zlokovic B V “Activated protein C enhances angiogenesis in brain endothelial cells in Alzheimer's disease” Soc. Neurosci. Abstract No. 9573 (2003); Chow N, Guo H, Zlokovic B V, “Senescence of cerebral endothelium in Alzheimer's Disease: An in vitro model system” Soc. Neurosci. Abstract No. 3897 (2003); and Sallstrom J F, Brooks A, Paxhia A, Song X, Guo H, Zlokovic B V, “Refinement of gene expression analysis in Alzheimer's Disease microvascular cells using laser capture microdissection” Soc. Neurosci. Abstract No.12271 (2003).

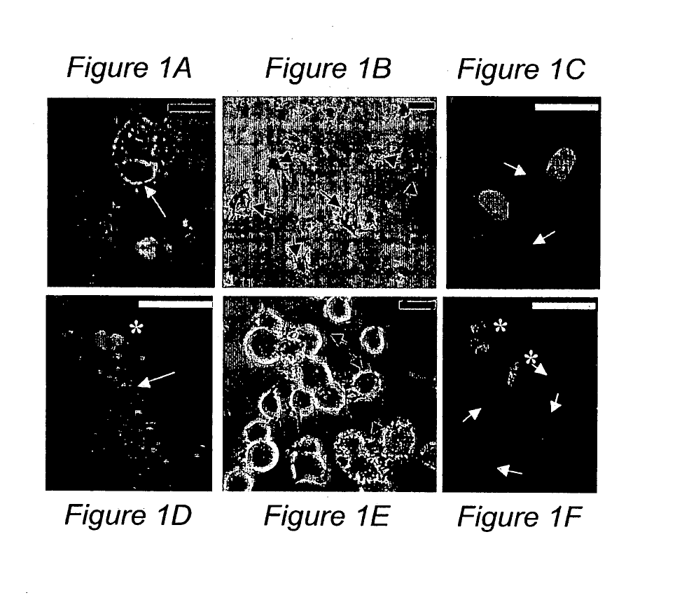

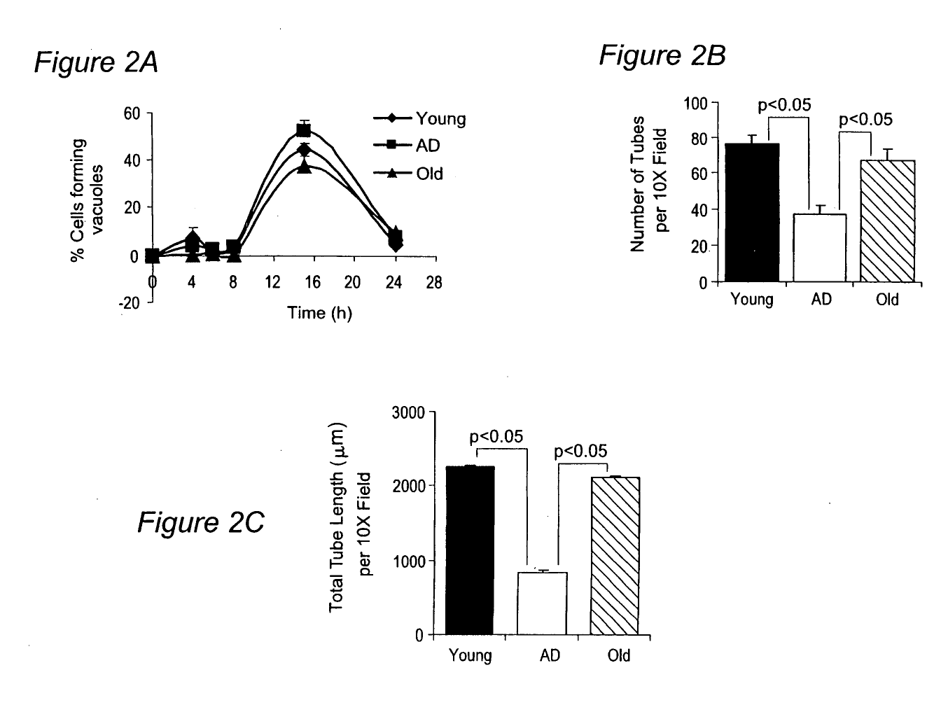

FIGS. 1-2 illustrate in vitro capillary morphogenesis in 3-D collagen matrices made by primary BEC (P2-P4) derived from six AD patients and six age-matched controls. The characteristics of patients and controls are given in Tables 1-2. As shown in FIGS. 1A-1B, BEC differentiation in controls begins with the formation of intracellular vacuoles (stage I) between 4 hr and 16 hr. No significant apoptosis is observed. Shortly after vacuolar stage, BEC elongate to form capillary tubes (FIG. 1C). At 24 hr, most BEC differentiate into capillary tubes (stage II; not shown). This model of BEC-mediated capillary morphogenesis in controls resembles that of systemic endothelial cells (Davis et al., J. Cell. Sci.114, 917-930, 2001), but some differences are discussed below.

In-contrast to control BEC, AD BEC exhibit early apoptotic changes in 20%-35% of the population. In the present AD model of brain capillary morphogenesis, a subpopulation of cells show blebbing of the cytoplasmic membrane (about 35%), chromatin condensation and/or nuclear fragmentation at 4 hr (FIGS. 1D-1E). Apoptosis of AD BEC could also be observed at 24 hr during capillary tube formation (FIG. 1F). Ultimately, AD BEC formed poor capillary networks compared to controls.

Counting of cells with vacuoles indicated no significant changes between AD and either of the two controls (FIG. 2A). But the number of tubes (FIG. 2B) and the total tube length (FIG. 2C) in the AD model at 24 hr were 2- to 2.7-fold less than in age-matched or young controls, respectively. This suggests that cells in AD die prematurely before the elongation into capillary tubes.

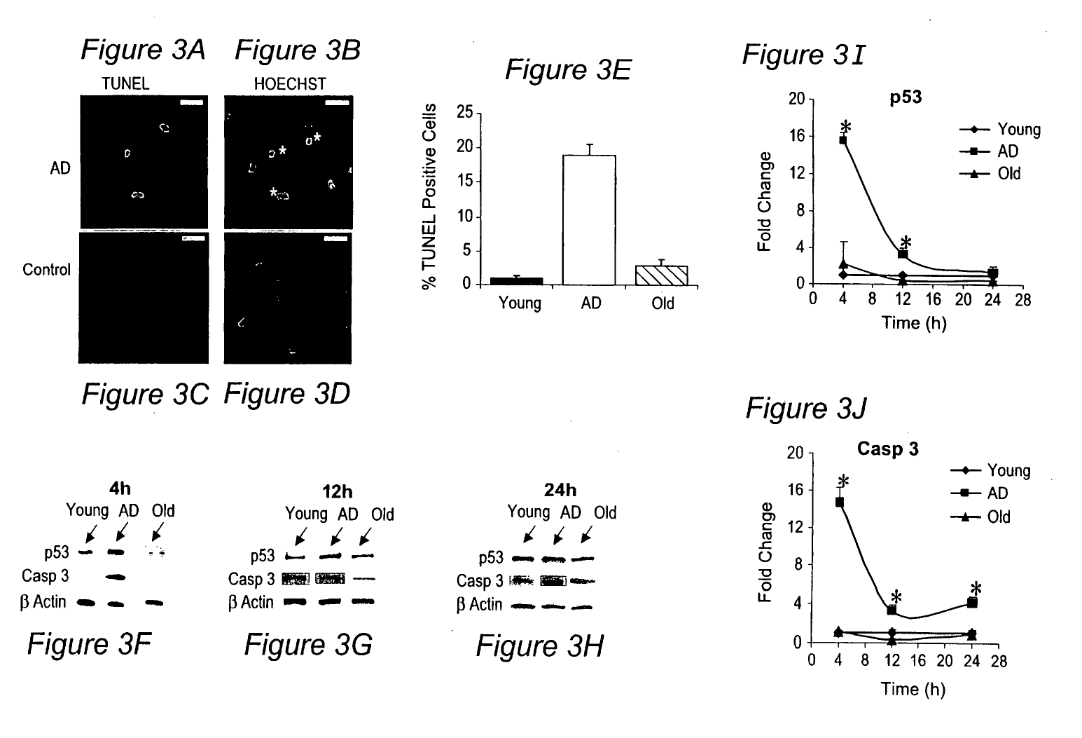

About 20% of cells in the AD model of brain capillary morphogenesis were TUNEL positive at 4 hr compared to either young or age-matched control cultures (FIGS. 3A-3E). To determine a possible mechanism for enhanced apoptosis in AD, Western blot analysis of cell lysates was performed (FIGS. 3F-3H): there was a significant increase in p53 in AD BEC compared to controls at 4 and 12 hr (FIG. 3I) and in caspase 3 in AD BEC throughout the different stages of capillary morphogenesis within 24 hr (FIG. 3J). The greatest increases of both p53 and caspase 3 were found during first 4 hr, which suggests a rapid activation of programmed cell death in AD BEC in response to angiogenic stimulation.

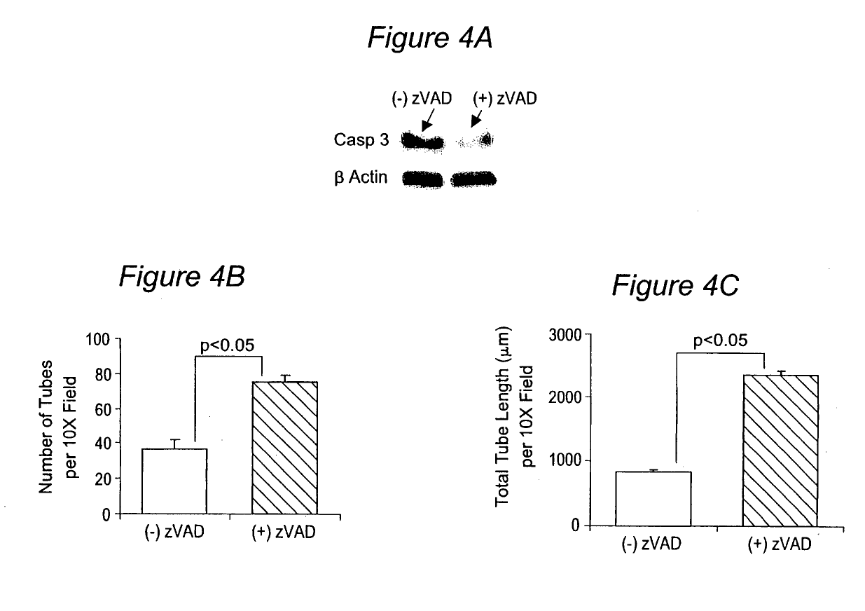

zVAD-fmk, a broad spectrum caspase inhibitor (Faleiro & Lazebnik, J. Cell Biol. 151, 951-959, 2000) was used. The results show that zVAD-fmk prevents apoptosis in AD BEC by preventing the formation of the active form of caspase 3 (FIG. 4A). Treatment with zVAD has resulted in the restoration of the number of capillary tubes (FIG. 4B) and total tube length at 24 hr (FIG. 4C) during AD BEC-mediated tubule formation. ZVAD also decreased a number of apoptotic AD BEC by TUNEL staining (not shown).

The morphological changes observed during in vitro angiogenesis assays correlated with the reported in situ observation in brains with Alzheimer's disease of atrophic vessels, blebbing of cytoplasmic membranes, capillary degeneration, tortuous vessels, regions of decreased microvascular density, and/or abnormal endothelial proliferation (Miyakawa et al., Virchows Arch. 40:121-129, 1982; de la Torre, Stroke 33:1152-1162, 2002). In contrast to the previous vascular theory's teaching that brain capillary degeneration is caused by hypoxia due to cerebral hypoperfusion in Alzheimer's disease (de la Torre, Neurobiol. Aging 21:331-242, 2000), the present findings suggest that the microvascular changes observed in brains of individuals with Alzheimer's disease could be due to defective angiogenesis and neovascularization caused by the inability of BEC to differentiate into capillary tubes, and/or an aberrant response of BEC to growth and angiogenic factors.

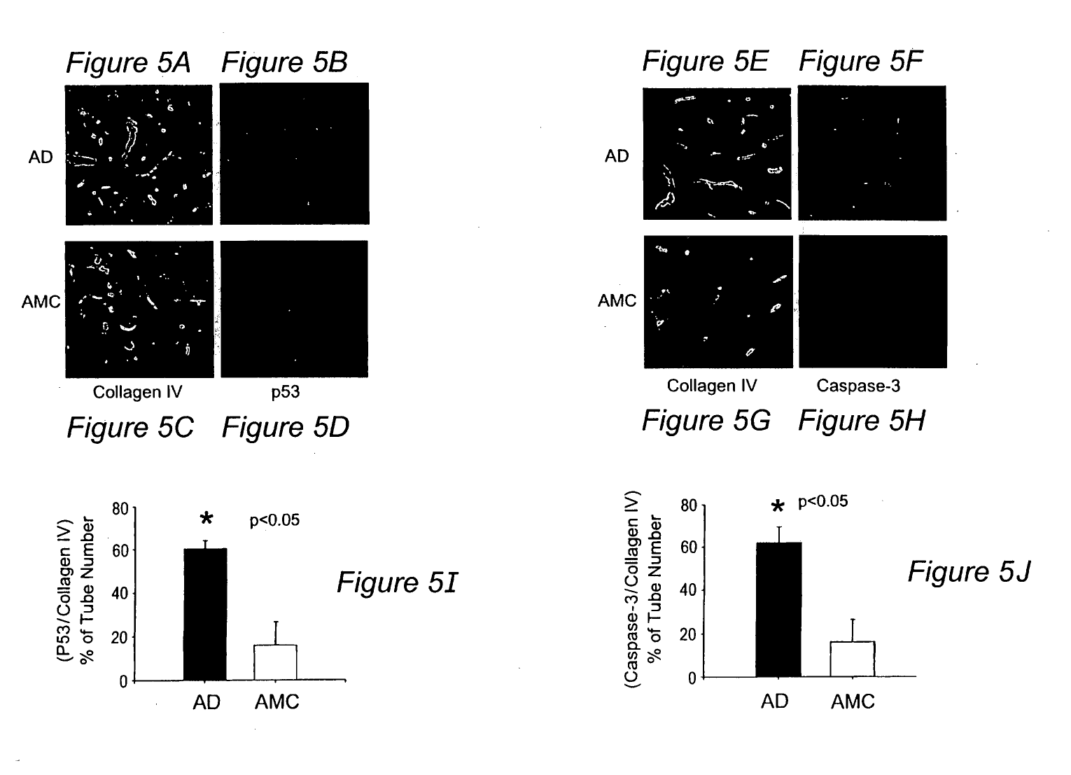

To further test whether similar molecular changes preceding apoptosis in our in vitro model also take place in brains in situ, immunodetection studies were performed for collagen IV (a vascular basement membrane marker) and p53 (FIGS. 5A-5D) and for collagen IV and caspase-3 (FIGS. 5E-H) on brain tissue sections Brodmann's areas 9 and 10 from AD patients and age-matched controls adjacent to the site of BEC isolation for in vitro studies (FIGS. 1-4). The in situ staining studies confirmed increased expression of p53-positive and caspase-3 positive brain microvessels in patients with AD compared to age-matched controls, i.e., about 60% of vessels were positive for both p53 and caspase-3 compared to less than 20% of positive vessels in controls (FIGS. 5I-5J). These studies are suggestive that p53-dependent apoptosis and caspase-3 pro-apoptotic signaling may take place in the part of the vascular system in AD brains in situ.

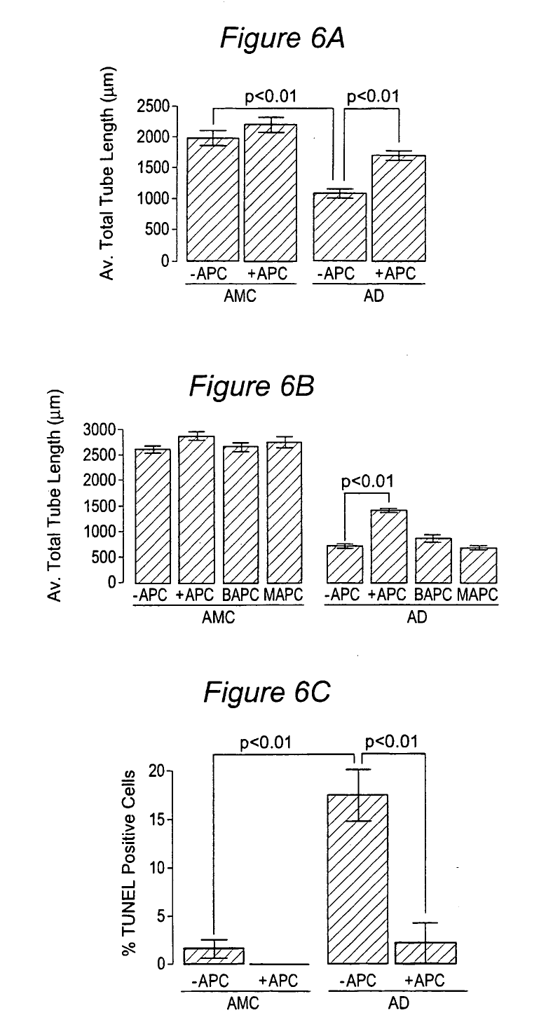

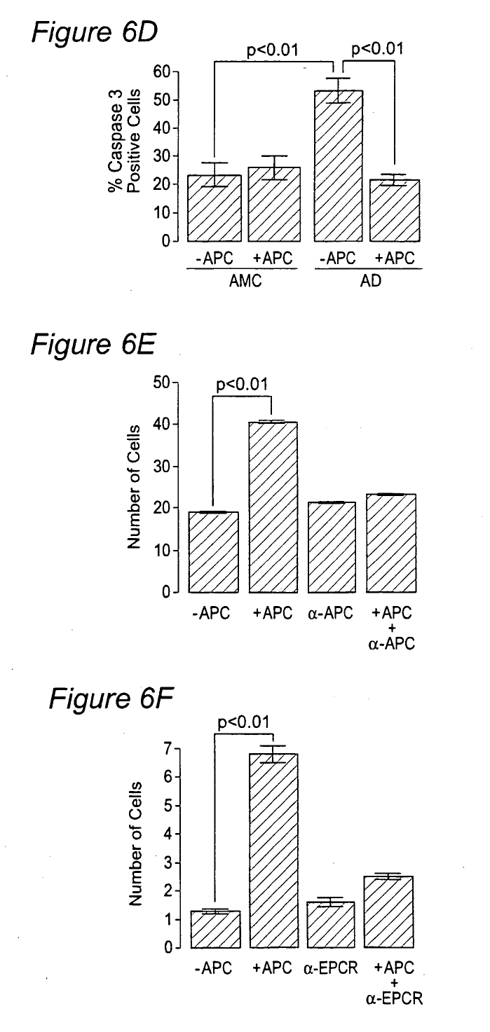

Next, studies with activated protein C (APC) were performed because APC has been recently reported to prevent apoptosis in human brain endothelium through endothelial protein C receptor (EPCR)-dependent activation of protease activated receptor-1 (PAR-1) by blocking p53 and caspase-3 pro-apoptotic signaling (Cheng et al., Nature Med. 9:338442, 2003). Microarray studies confirmed the presence of both EPCR and PAR-1 mRNA in control and AD BEC, and no changes in the level of their transcripts in AD BEC vs. control BEC (not shown). FIG. 6A illustrates that APC (100 nM) significantly improved AD BEC-mediated formation of capillary tubes by greater than 50% and that the active catalytic site serine is required for this effect, which suggests the possible involvement of PARs (FIG. 6B). As a negative control, heat-inactivated APC was without effect (FIG. 6B). APC significantly reduced the number of TUNEL-positive cells and caspase-3 positive cells during AD BEC-mediated brain capillary morphogenesis by 84% and 61%, respectively (FIGS. 6C-6D), which confirms the significant anti-apoptotic activity during AD BEC-mediated tubule formation. APC (100 nM) also enhanced migration of AD BEC by>50% (FIG. 6E). This effect was neutralized by monoclonal C3 anti-APC antibody (Heeb et al., Thromb. Res. 52:3343, 1988). EPCR appears to be required for this effect of APC (FIG. 6F).

The pattern of capillary morphogenesis in control BEC is similar to HUVEC (Davis et al., J. Cell Sci. 114:917-930, 2001), but there was a significant difference in the time course of formation of the intracellular vacuoles: 12 hr to 16 hr for BEC compared to 4 hr to 8 hr for HUVEC. It is noteworthy that BEC exhibit hairy cytoplasmic processes and their cell bodies become positive for neuronal markers at early stages of differentiation (i.e., between 1 hr and 4 hr) as during early stages of neurogenesis (e.g., neuron-specific tubulin TuJ, tyrosine hydroxylase, etc.). This was never seen in other endothelial cells including HUVEC. In addition, a significant number of BEC at this early stage (1 hr to 4 hr) were able to acquire the morphological appearance of neurons, showing processes that resemble neuritic or dendritic processes, and the shape of cells that resembles the shape of pyramidal cells in the brain, bipolar cells in the retinal ganglion, and/or rounded granular cells from the cerebellum. These findings raise the possibility that primary BEC may preserve some of the features of pluripotent precursor cells and could hold eventually a potential to differentiate towards the neuronal lineage if cultured in the environment suitable for neuronal growth and differentiation and in the presence of neuronal growth factors.

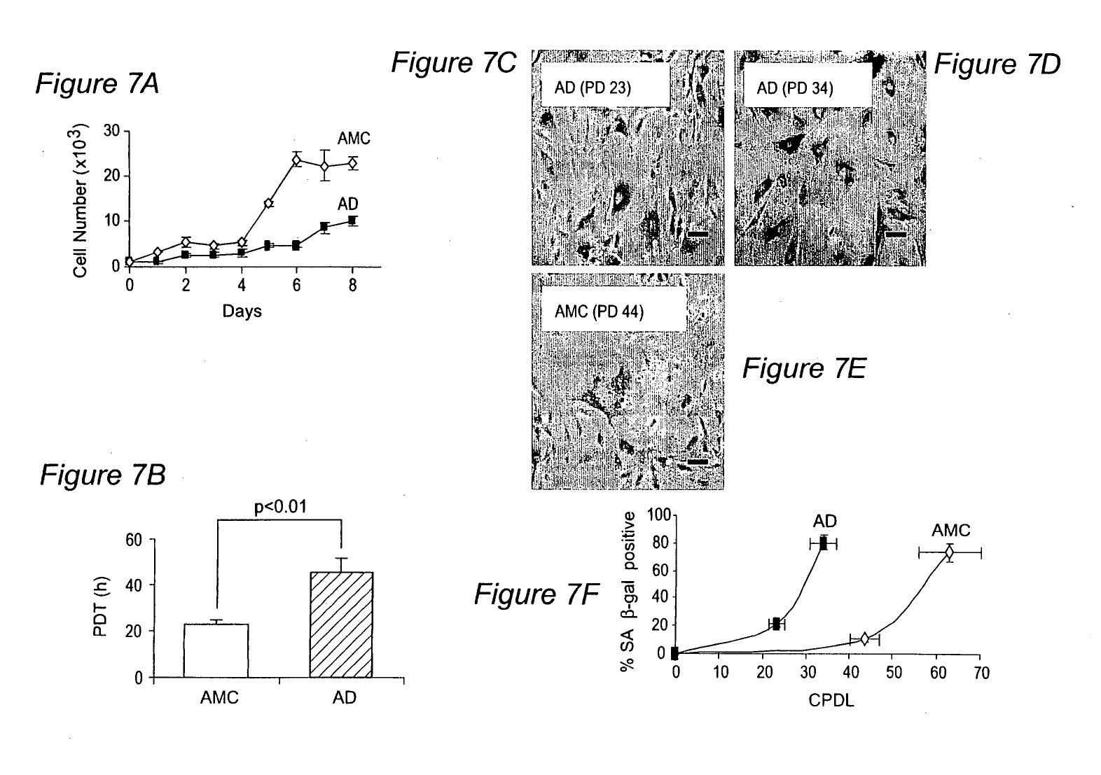

A subpopulation of BEC derived from Alzheimer's disease patients, in addition to those actively dividing and dying by apoptosis, include senescent cells. FIG. 7A shows impaired growth of AD BEC compared to control BEC; their population doubling time (PDT) was longer by 2-fold than for age-matched controls (FIG. 7B). AD BEC compared to control BEC at earlier passages express common markers of senescence including enlarged and flattened morphology and expression of senescence-associated-β-galactosidase (SA-β-gal) at pH 6.0 (Campisi et al., Exp. Gerontol. 31:7-12, 1996). There was a progressive increase in number of SA-β-gal-positive senescent cells in AD BEC from 23 PD (FIG. 7C) to 34 PD (FIG. 7D) when most cells become senescent. On the other hand, AMC BEC cultured under same conditions have insignificant number of senescent BEC even after 44 PD (FIG. 7E). FIG. 7F shows that AD BEC reach a stage of almost complete replicative senescence after about 30 cumulative PDs, while AMC BEC at that stage express a negligible number of senescent cells. The senescent phenotype was further revealed by deficits in early- and mid-G1 phase in response to serum stimulation in AD BEC (not shown), and increased expression of p16, both at the transcript and protein level (see below). Thus, it appears that replicative senescence of AD BEC in culture reflects senescence of the vascular system in Alzheimer's disease brains, and development of prematurely-aged dysfunctional vasculature as suggested by molecular analysis (see below).

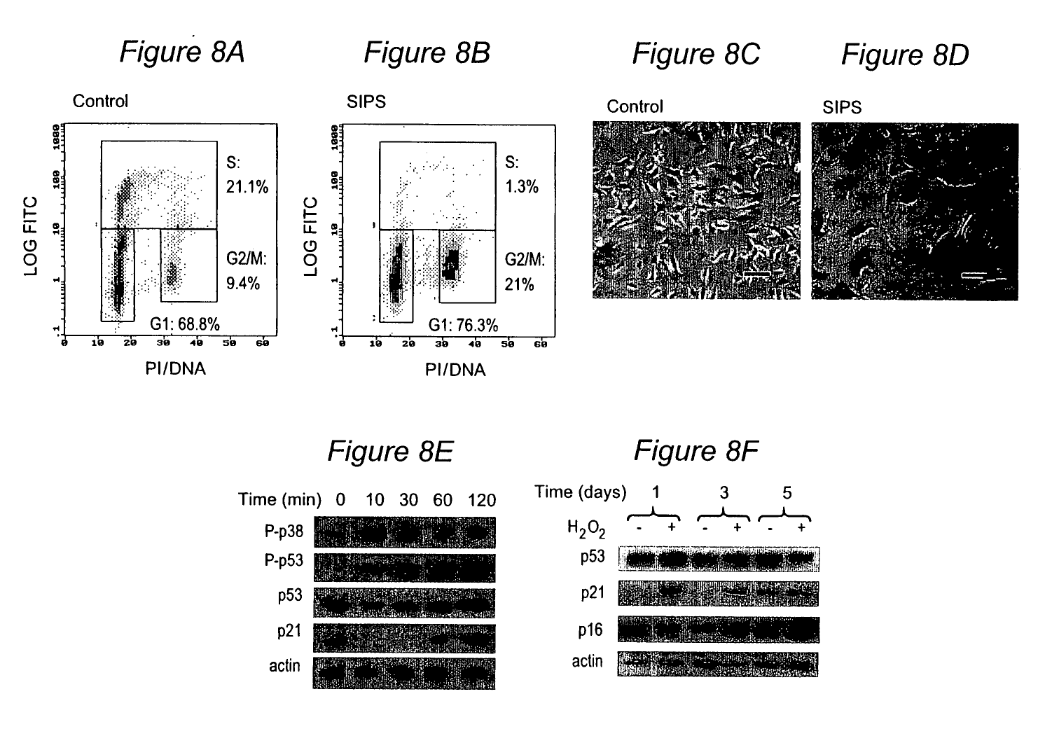

To better understand the molecular basis of AD BEC senescence and to develop a simple model to test different therapeutic strategies for AD vascular disorder, an H₂O₂ model of SIPS (Chen et al., J. Cell Sci. 11 3:4087-4097, 2000) was adapted to human BEC. BEC treated with sublethal concentrations of H₂O₂ (300 μM) for 2 hr developed a senescent phenotype within 48 hr with all of the phentotypic markers characteristic of senescence, including G1 cell arrest (FIGS. 8A-8B), enlarged and flattened morphology, and expression of SA-β-gal at pH 6.0 (FIGS. 8C-8D). The concentration of H₂O₂ that induces BEC senescence was determined after initial studies with different concentrations of H₂O₂ from 10 μM to 1 mM and by comparing its toxic effect (e.g., LDH release, nuclear condensation, and fragmentation) to its effect on cell cycle and mitosis. These studies revealed that control BEC treated with 300 μM H₂O₂ develop a phenotype indistinguishable from those of senescent BEC derived from individuals with Alzheimer's disease. Higher concentrations of H₂O₂ resulted in BEC apoptosis (not shown).

H₂O₂-treated cells became senescent through a series of molecular events which included changes in the expression of cell cycle regulator genes. The first noticeable early changes were transient p38 phosphorylation and p53 phosphorylation within 10 min and 30 min of exposure to H₂O₂, respectively, followed by induction of cyclin-dependent kinase inhibitor (CDKI) p21^{CIP1} (FIG. 8E) that peaked approximately at 48 hr and remained elevated within next 48 hr to 72 hr (FIG. 8F). Finally, there was a progressive elevation of CDKI p16^{INK}^{4a} that stabilized at 72 hr (FIG. 8F) and remained elevated for the remaining studied period of several days (Chow et al., Soc. Neurosci. Abstract No. 420.9, 2002). At 24 hr, there was also lack of Rb phosphorylation. All these changes resulted in withdrawal of brain endothelial cell from the cell cycle, permanent G1 arrest as shown in FIGS. 8A-8B, and development of the senescent phenotype (FIGS. 8C-8D). H₂O₂-treated cells had higher levels of fibronectin and PAI-1 (not shown), markers usually associated with senescence of fibroblasts (Chen et al., J. Cell Sci. 113:4087-4097, 2000).

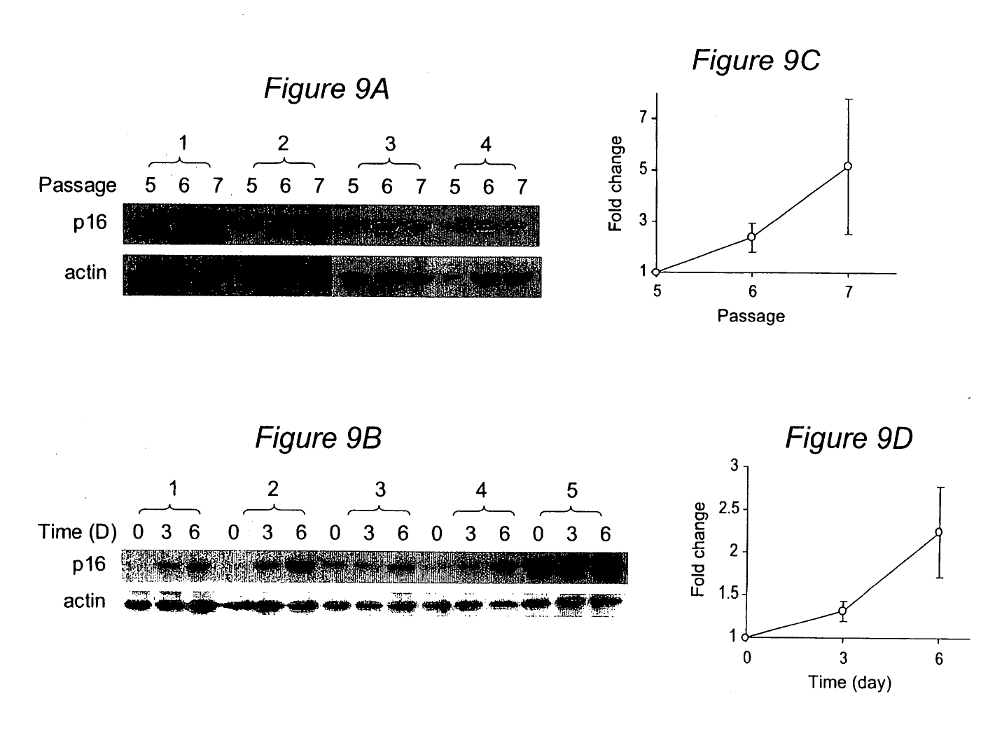

Since p16 has been shown to play a critical role in stabilizing endothelial cell senescent phenotype (Shelton et al., Current Biol. 9:939-945,1999; Choi et al., FASEB J. 15:1014-1020, 2001), p16 expression during replicative senescence in AD BEC and after SIPS in control young BEC was determined by Western blot analysis. FIGS. 9A and 9C show a time-dependent progressive increase in p16 expression with each increasing passage of AD BEC (i.e., shown from passage 5 to passage 7). FIGS. 9B and 9D confirm the progressive increase in p16 levels in a H₂O₂ model of BEC SIPS from 0, 3 and 6 days. These findings confirmed that in both RS AD BEC and SIPS BEC, p16 is critically associated with a senescent phenotype.

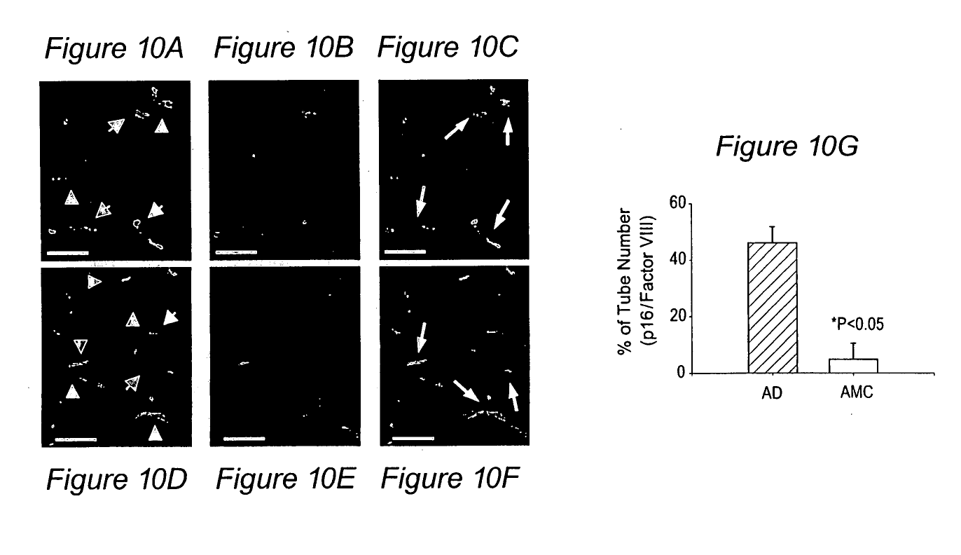

The analysis of brain tissue confirmed increased number of p16-positive vascular profiles in AD compared to age-matched controls (FIGS. 10A-10F). The number of p16-positive brain vessels in AD was about 40% compared to 5% in controls (FIG. 10G), which suggests that there is vascular senescence in AD brain endothelium in situ. Double staining with CD-105 (endoglin), a marker for the abluminal site of the endothelium in situ, and CD31 (PECAM), a marker for the luminal site of the endothelium in situ, has revealed flattening of the cell cytoplasm in AD BEC in tissue resembling the senescent changes seen in vitro with the isolated cells. Loss of cell shape due to loss of stabilizing cytoskeletal elements can make cells prone to mechanical injury and may obstruct the lumen of microvessels compromising brain circulation, CBF regulation and BBB transport.

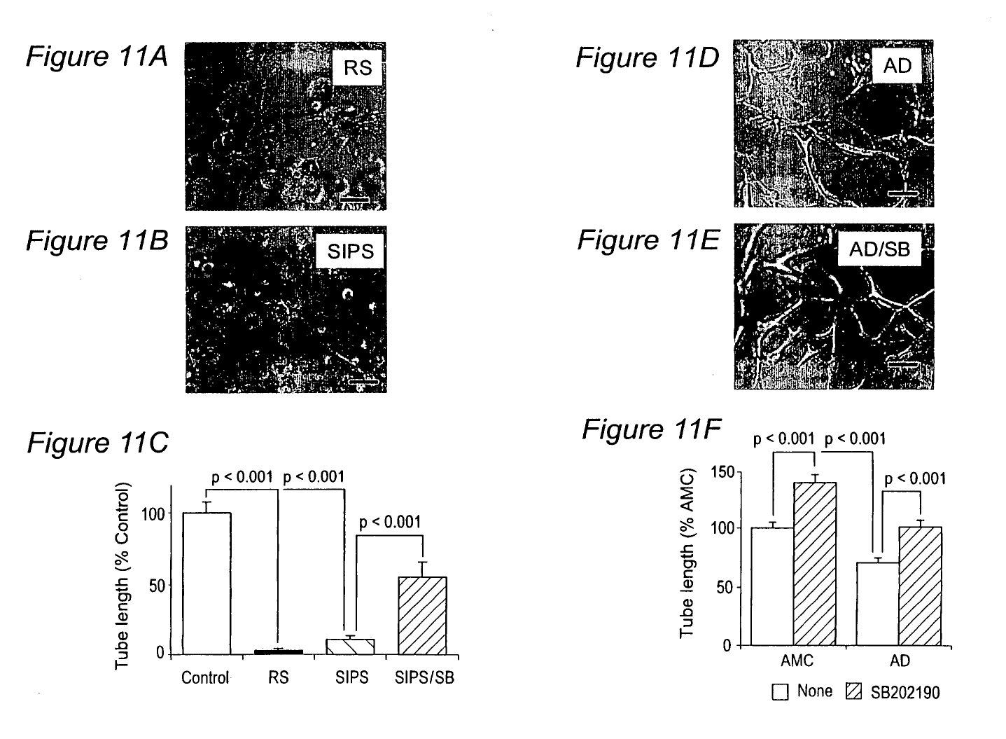

H₂O₂-treatment resulted in early transitory phosphorylation of p38 MAPK that peaked at 1 hr to 2 hr, and returned to normal levels shortly after that. It has been proposed that activation of p38 MAPK may control apoptosis during angiogenesis thus playing a crucial role in vascular remodeling, as for example during FGF-mediated angiogenesis (Mastumoto et al., J. Cell Biol. 156:149-166, 2002). Therefore, the performance of H₂O₂-treated BEC and RS BEC was assayed for capillary morphogenesis. FIGS. 11A-11C show that both SIPS BEC or RS BEC form an insignificant number of tubes compared to untreated normal control cells. But preincubation of BEC with SB202190, a p38 MAPK activation inhibitor, significantly improved tube formation in the SIPS model augmenting total tube length by almost 5-fold (FIG. 11C). Moreover, treatment of early passage AD BEC with SB202190 completely restored the number of tubes to the level of control BEC (FIG. 11D-11F). Thus, an early activation of p38 MAPK in response to angiogenic signaling in SIPS BEC or AD BEC may lead to massive apoptosis and an imbalance between formation and regression of new vessels in diseases such as Alzheimer's disease vascular disorder and/or pre-senescent and senescent state of brain endothelial cell.

To further understand molecular events involved in abnormal responses to angiogenic signaling and aberrant angiogenesis in AD BEC and cellular senescence, we performed several high-throughput screening microarray studies on BEC (P2-P4) from patients and controls were performed.

In the first analysis (Table 9), changes in gene expression were compared between six individuals with AD and six age-matched or five young controls on Affymetrix U95A chips (12,500 genes). The average age of AD and age-matched controls was about 70 years. There was no significant differences in gender ratio, the PMI, and cause of death. The incidence of vascular risk factors was 67% in either group. But AD cases had significantly higher incidence of CAA, i.e., 6/6 (100%) vs. 2/6 (33%) than controls. AD cases were Braak stage V-VI compared to 0 or 0-I in controls, and CERAD 50/50 moderate to frequent compared to negative or sparse in controls. The average CDR in this AD group was close to 4, while it was zero in age-matched controls. Detailed characteristics of the groups are given in Tables 1-3.

In the second analysis (Tables 10-11), changes in gene expression were compared between 11 individuals with AD and five age-matched, five middle-age, or five young controls on Affymetrix U133A and U133B chips (45,000 genes). The average age of AD and age-matched controls was about 80 years. There was no significant differences in gender ratio, PMI, and cause of death. The incidence of vascular risk factors was high and comparable in both groups, AD (73%) and age-matched controls (80%). The incidence of CAA was 82% in AD and somewhat lower in age-matched controls (60%). AD cases were all Braak stage V-VI compared to 0-I or I-Il in controls. By CERAD AD were 72/28 frequent to mode-rate, while age-matched controls were sparse (60%) to moderate (40%). The average CDR in this AD group was also close to 4, while it was zero in age-matched controls. Detailed characteristics of the groups are given in Tables 4-8.

In the third analysis (Table 12), the coincidence genes were studied with Affymetrix U95A chips between RS-AD BEC vs. early passage AD BEC in three AD cases compared to SIPS young BEC (four cases) vs. control young BEC (five cases).

Bayesian correction for data analysis of the Affymetrix U95A, U133A, and U133B array results used the criteria described (i.e., 2-fold ratio, expression 500, 0.05 Bayesian p-log).

The first analysis (Table 9) has revealed significant differences in expression in a subset of 42 genes (i.e., 0.3% out of 12,500) in Alzheimer's disease compared to age-matched controls (p<0.05 or the difference is found in greater than 95% of cases). In AD, several transcription factors and genes with predicted actions in cell differentiation and angiogenesis, signal transduction, cytoskeleton, matrix, and cell cycle regulation were significantly dysregulated compared to age-matched controls (Table 9). No changes in the expression of these genes were observed between young and age-matched controls with the exception of L-3-phopshoserine phosphatase that was significantly increased in age-matched vs. young BEC and exhibited further sharp increase in AD vs. age-matched controls. This suggests that reported gene expression profile in AD was largely age-independent.

An independent validation of microarray data was performed by QRT-PCR analysis for a subset of genes including gax, tissue transglutaminase (TG2), eukaryotic translation initiation factor 2 gamma subunit (elF2γ) and asparaginyl tRNA synthetase, a gene that was not altered in AD vs. controls by Bayesian analysis (1.007-fold difference; p=0.91). The QRT-PCR analysis confirmed the same direction of change for gax, TG2 and elF2γ, and the magnitude of change was even more significant than by the microarray analysis, i.e., by −16.7-fold, +14.8-fold and −10.1-fold, respectively, and no change in the expression of asparaginyl tRNA synthetase.

The homeobox gene gax (Gorski & Walsh, Circ. Res. 87:865-872, 2000) was down regulated 2-fold in AD BEC. Down regulation of gax mRNA and gax homeoprotein was confirmed by QRT-PCR analysis (−16.7-fold) and Western blot analysis of cell lysates (−3.7-fold). It was also undetectable in brain microvessels in tissue sections from brains of AD patients, but was expressed in brain microvessels in age-matched and/or young controls (not shown). Consistent with the prediction that down regulation of gax would lead to up regulation of αvβ3 and αvβ5 integrins (Witzenbilcher et al, J. Clin. Invest. 104:1469-1480, 1999), it was confirmed by FACS analysis that BEC of individuals with AD express consistently higher levels of cell surface αvβ3 and αvβ5 integrins, but not the αvβiintegrins. The migration of AD vs. control BEC in a Boyden chamber indicated that AD BEC migrate by about 2-fold slower than controls. Increased levels of αvβ3 and αvβ5 integrins, but again not the αvβ1 integrins, have been confirmed in brain endothelium in tissue sections of individuals with AD for the sites adjacent to the site used for BEC isolation.

Although some of these genes (Table 9) have functions relevant to cell growth and differentiation, apoptosis, and specialized functions of the vasculature their effects on the biology of BEC in relation to a neurodegenerative disorder have not been previously demonstrated. The altered BEC physiology is at least in part manifested in dysregulation of the brain vasculature (e.g., abnormal angiogenic. signaling, activation of programmed cell death, aberrant angiogenesis and/or cellular senescence). Here, it is shown that the differences in gene expression can be related to changes in the cell biology of the vasculature in patients (i.e., Alzheimer's phenotype) and function of the BBB. Anatomic and enzymatic components of the BBB are reviewed in McComb and Zlokovic (Cerebrospinal fluid and the blood-brain interface, In: Textbook of Pediatric Neurosurgery, Philadelphia, Pa.: Saunders, 2000).

The functions discussed here for brain endothelial cells may act independently, additively, or synergistically in Alzheimer's disease: loss of neurotrophic support, reduced detoxification, dysregulation of cell growth in the microvasculature (e.g., smooth muscle, endothelial cell) leading to nonsense angiogenesis and incompetent capillary morphogenesis. This defect may also either reduce the longevity of the vascular system in the brain and/or predispose it to SIPS. The presence of senescent cells in the vascular system can significantly reduce normal physiological functions of the BBB related to molecular exchange between blood and brain and may impair the CBF regulations. Thus, dysregulation of growth and senescence of BEC, can be linked to pathogenesis of Alzheimer's disease and mechanisms of disease.

These discoveries shift attention in understanding Alzheimer's disease from plaque formation in the neuronal and vascular compartments to the microvasculature that comprises the blood-brain barrier. The phenotypic drift and destabilized gene expression profile of endothelium may result from a disease-specific defect primary to the vascular system. This can have consequences for pathophysiology of the brain vascular system leading to clinical dementia. It is envisioned that these pathogenic pathways may be coordinated by “master” key genes which regulate one or more of the pathways. It is also possible that the primary defect is caused by somatic mutation and/or chromosomal translocation in brain endothelium that inhibits normal responses of BEC to angiogenic signaling and differentiation into functional capillary tubes. Western blot analysis of cell lysates, ELISA of cell culture supernatants, and/or immunocytochemical analysis of brain tissue in situ for several gene products suggest a general and consistent agreement with oligonucleotide array results. Proteomic studies (e.g., quantitative or semiquantitative Western blotting, ELISA, and immunostaining) have confirmed that the changes in gene expression observed as RNA transcribed for a subset of genes are also detectable at the level of translated protein. In general, the direction of the change in gene expression (i.e., increased or decreased) is the same but the magnitude of any difference may vary. This may reflect differences in the cell cultures or samples obtained therefrom, regulation at the level of protein translation or processing, saturation of the protein translation or protein processing, or the like.

These findings further indicate that correcting defective differentiation of brain endothelial cells into vascular tubes could be a distinct therapeutic target in Alzheimer's disease. Several gene and molecular candidates underlying these cellular defects are described below. Preventing programmed cell death of brain endothelium which is stimulated by growth factors and/or during an angiogenic response is clearly a distinct vascular therapeutic target in Alzheimer's disease. Examples of drugs that are able to prevent and/or reverse aberrant AD BEC-mediated angiogenesis or angiogenesis from senescent BEC are given in FIGS. 4, 6 and 11, with zVAD, APC and p38 MAPK inhibitor, respectively.

Here, we describe in greater detail changes in gene expression that are statistically significant and therefore considered to be present in greater than 95% of Alzheimer's disease cases compared to age-matched controls in the first analysis (Table 9) of cases listed in Tables 1-3. The reference list is given below.

Based on expression profiling, the role of gax, a transcription factor which is involved in angiogenesis, vascular remodeling, and regulation of the cell cycle and cell migration (1), was determined. The gax homeoprotein is down regulated in AD brains in situ, which validates the present AD BEC model of defective angiogenesis (Hofman et al., Soc. Neurosci, Abstract No. 328.3, 2002). Bearing in mind the importance of gax-integrin axis (2), the regulation of αvβ3 and αvβ5 integrins in AD BEC and in microvessels of AD brains in situ was analyzed, and the results confirmed a significantly increased cell surface expression of these integrins in AD BEC in vitro by 5-fold and 3-fold, respectively, and in brain vessels in AD brains in situ by 4-fold and 55%, respectively, based on the number of positive vascular profiles. These data again, validate the importance of the gax-integrin axis in our in vitro BEC model.

αvβ3 and αvβ5 interins play a critical role in angiogenic signaling, cell migration and proliferation (3). In previously reported studies, up regulation of αvβ3 and αvβ5 integrins in response to gax down regulation was linked to increased cell migration (2). In the results presented here, upregulated integrin expression led to decreased migration. This suggests that the gax-integrin interactions may be cell specific, these interactions are aberrant in AD BEC, and/or the result reflects the activation of a compensatory program that attempts to balance an as-of-yet unidentified defect in angiogenesis in the AD cells.

Cell death during brain capillary morphogenesis in AD may result from increased levels of p53 as shown for different cell types including endothelium (4). Since AD cells migrated significantly slower than controls, the increased αvβ3 and αvβ5 integrins expression in AD BEC is most likely functioning aberrantly, either with decreased integrin affinity or avidity, or abnormal function (e.g., integrin detachment function that may favor cell death in AD by anoikis (3,5). The relationship between integrin activity and p53 is not well understood, but it has been reported blocking and/or aberrant integrin signaling may trigger apoptosis by activating p53 (6). A stabilization of p53 at Ser15 phosphorylation site of ataxia teleangiectasia mutated kinase in response to DNA damage (7) was not found in the present study (not shown). However, DNA damage still may take place in AD BEC as suggested by up regulation of AP endonuclease XTH2 gene (Table 9), a nuclear and mitochrondrial DNA repair enzyme (8).

The present gene array analysis strengthens the hypothesis that brain capillary degeneration in AD is independent of hypoxia, by demonstrating no changes in hypoxia-inducible genes, e.g., the hypoxia-inducible factor 1 (HIF-1) gene and its HIF-1α subunit, a transcription factor which is induced by decreased cellular oxygen (9). These gene array data are also confirmed by the Western blot analysis (not shown). The expression of VEGF receptors, VEGFR-1 (Flt-1) and VEGFR-2 (Flk-1/KDR) and tyrosine kinase receptors implicated in angiogenic signaling (e.g., tie-2, tie-1, FGF receptors) (10,11) was not significantly altered in AD BEC according to the microarray data, and confirmed for some by FACS analysis (e.g., VEGFR-1, VEGFR-2) and Western blot analysis of cell lysates (not shown).

Altered expression of several genes in AD BEC may contribute to impaired signal transduction. Sharply increased expression of L-3-phosphoserine-phosphatase (Table 9) can counteract the activity of serine/threonine kinases and increase cerebrovascular levels of D-serine which blocks the synaptic transmission via N-methyl-D-aspartate glycine receptor (12). Down regulation of calcium, calmodulin regulated 3,5-cyclic nucleotide phosphodiesterase will lead to accumulation of cyclic adenosine mono phosphate (13). Down regulation of the gene encoding REM, a member of subfamily of RAS-related GTPases that include Rad, Gem, and Kir (14), and up regulation of the gene encoding Nef-associated factor 1 beta that associates with several cellular tyrosine kinases and serine/threonine kinases (15) can also modify signal transduction.

Ankyrin G (ANK-3) is significantly down regulated in AD BEC (Table 9). Ankyrins represent a protein family whose members are associated with membrane proteins and the actin cytoskeleton, and are involved in regulating several cellular processes (16). Integrin-linked kinase (ILK), a multidomain focal adhesion protein that is involved in adhesion of cells to the matrix and signal transduction mediated by integrins, binds with high affinity to PINCH, a focal adhesion protein, via the N-terminal ankyrin repeat domain (16). So although down regulation of ankyrin G might not be specific for the malfunction of ILK in BEC of individuals with AD, it may contribute to deficient signal transduction observed in these cells.

Gene array analysis indicated down regulation of TINUR NGF-B/nur 77 beta type transcription factor, a member of the steroid/thyroid hormone nuclear receptor superfamily (17), which predicts a deficient response of AD BEC to thyroid/steroid hormones during differentiation. C-maf proto-oncogene, a member of a large family of basic zipper transcription factors (18) is also down regulated in AD. C-maf can form heterodimers with jun and fos and may induce cell death through its control of p53 expression (18). The AFX forkhead transcription factor is 2-fold increased in AD BEC. Studies have shown that the AFX forkhead transcription factor directly regulates apoptosis by suppressing the anti-apoptotic BCL-XL protein (19), that is also down regulated in AD BEC (not shown). Increased levels of BAI2, a p53-target gene homologous to brain-specific angiogenesis inhibitor 1 (20) could modulate pathogenic response of AD BEC during angiogenesis.

Increased brain endothelial expression of tissue transglutaminase T2, an enzyme that catalyzes varepsilon-lysine to gamma glutaminyl isodipeptide bonds, has been reported in AD brains (21). Transglutaminase is involved in cross-linking of tau and neurofilaments in AD. Our studies in brain tissue sections confirmed its endothelial localization (data not shown) suggesting that endothelium may be an important source of increased transglutaminase activity in AD brains.

The key translation initiation factor 2 (elF2) gamma subunit (22), and a ribosomal protein 37a, encoding a ribosomal component of the 60S subunit were down regulated in AD BEC, which predicts reduced protein synthesis overall. AD BEC down regulate plectin, a member of the cytolinker family, which is a stabilizing element of cells against mechanical stress and is a substrate of caspase 8, that may be required for reorganization of the microfilament system during apoptosis (23). Down regulation of procollagen I-N proteinase, a metalloproteinase that cleaves amino-propetides in the processing of type I procollagen into collagen (24) predicts defective matrix processing during AD-mediated angiogenesis.

It is intriguing that the present analysis revealed that the gene encoding MTG8-related protein MTG16a was significantly up regulated in AD BEC. This gene was originally identified as one of the loci involved in t(8;21)(q22;q22) chromosomal translocation in acute myeloid leukemia (25). Its role in AD BEC vascular pathology remains to be determined. One or more master key genes may initiate genomic instability in AD BEC possibly by inducing somatic mutations in the endothelium.

It is noteworthy that about 7.5% of dividing early passage subconfluent BEC derived from individuals with Alzheimer's disease had irregular nuclear boundaries, multi-lobed nuclei, and multiple nucleations, which are characteristic of mitotic catastrophe (Jonathan et al., Curr. Opin. Chem. Biol. 3:77-83, 1999). Cells undergo mitotic catastrophe if their chromosomal DNA is damaged (e.g., by irradiation). Such cells cannot successfully complete the cell cycle as they cannot enter mitosis, and therefore they die by mitotic catastrophe. This type of cell death is different from classically-described apoptosis and is characterized by the inability of the cells to divide (although their nuclei can divide, so typically one finds multinucleation), and they eventually die. An increased index of mitotic catastrophe in Alzheimer's disease cells is found when they are stimulated to grow by growth factors.

Mitotic catastrophe is frequently associated with increased expression of proteins involved in the initiation and execution of mitosis. Transcriptional profiles suggest significant down regulation of several tumor suppressor genes including gax (−2-fold; p=0.003), interferon inducible protein 9-27 (−2.2-fold; p=0.04), growth arrest specific gene 1 or gas1 (−2.6-fold; p -0.14), AIM-1 (−3.0-fold; p=0.12), c-maf (−3.1-fold; p=0.005), a transcription factor that has the potential to act as tumor suppressor, and increased expression of PISSLRE gene or CDK10, a novel member of cdk family implicated in the G2/M transition (+2.5-fold; p=0.01). Increased activity of MTG-8 may lead to a hyperproliferative response as well.

Thus, mitotic catastrophe could be one possible way to remove damaged brain endothelial cell from the vascular system in Alzheimer's disease patients. On the other hand, brain endothelial cells in Alzheimer's disease may activate a cellular program for senescence. Decreased expression of gax (1,2) and significantly decreased expression of the antiproliferative interferon-inducible protein 9-27 gene (26) demonstrate that there are cell cycle abnormalities in AD BEC.

Cell death (through mitotic catastrophe followed by apoptosis) and senescence are two independent responses that could be co-induced by different types of cellular damage (Jonathan et al., Curr. Opin. Chem. Biol. 3:77-83, 1999). In contrast to replicative senescence, stress-induced premature senescence (SIPS) in AD BEC could reflect a programmed protective response to cellular stress as in other age-related diseases (e.g., atherosclerosis, diabetes, and the more general problem of organismic aging).

Significantly slower protein synthesis is suggested by down regulation of some key components, such as elF2 gamma subunit and ribosomal protein 37, along with overexpression of transglutaminase TG2, which is likely to reflect the presence of senescent cells in AD BEC population. Up regulated in BEC of individuals with Alzheimer's disease is cytochrome P450C11 beta, CYP11B1 (+2.5-fold, p=0.005) also increased in senescent HUVEC. It is noteworthy that the AD BEC in the first analysis were from early passages that contain approximately 20%-40% of senescent cells. As shown in Table 12, the molecular and cellular phenotypes of the BEC population which contain greater than 85% replicative senescent cells are very different from mixed AD BEC populations with lower numbers of senescent cells, but is similar to the profile of SIPS in control BEC, as discussed below.

References

1. Gorski & Walsh (2000) The role of homeobox genes in vascular remodeling and angiogenesis. Circ. Res. 87:865-872.

2. Witzenbichler et al. (1999) Regulation of smooth muscle cell migration and integrin expression by the Gax transcription factor. J. Clin. Invest. 104:1469-1480.

3. Smyth & Patterson (2002) Tiny dancers: The integrin-growth factor nexus in angiogenic signaling. J. Cell Biol. 158:17-21.

4. Oren (1999) Regulation of the p53 tumor suppressor protein. J. Biol. Chem. 274:36031-3603.

5. Frisch & Ruoslahti (1997) Integrins and anoikis. Curr. Opin. Cell Biol. 9:701-706.

6. Lewis et al. (2002) Intergrins regulate the apoptotic response to DNA damage through modulation of p53. Proc. Natl. Acad. Sci. USA 99:3627-3632.

7. Carr (2000) Piecing together the p53 puzzle. Science 287:1765-1766.

8. Tsuchimoto et al. (2001) Human APE2 protein is mostly localized in the nuclei and to some extent in the mitochondria, while nuclear APE2 is partly associated with proliferating cell nuclear antigen. Nucl. Acids Res. 29:2349-2360.

9. Semenza (1998) Hypoxia-inducible factor 1: Master regulator of O₂ homeostasis. Curr. Opin. Gen. and Dev. 8:588-594.

10. Yancopoulos et al. (2000) Vascular-specific growth factors and blood vessel formation. Nature 8:242-248.

11. Qarmeliet (2000) Mechanisms of angiogenesis and arteriogenesis. Nature Med. 6:389-395.

12. Kumashiro etal. (1995) Free D-serine in post-mortem brains and spinal cords of individuals with and without neuropsychiatric diseases. Brain Res. 681:117-125.

13. Kakkar et al. (1999) Clamodulin-dependent cyclic nucleotide phosphodiesterase (PDE1). Cell Mol. Life Sci. 55:1164-1186.

14. Finlin & Andres (1997) Rem is a new member of the rad- and gem/kir ras-related GTP-binding protein family represed by lipopolysaccharide stimulation. J. Biol. Chem. 272:21982-21988.

15. Fukushi et al. (1999) Identification and cloning of a novel cellular protein Nef1, Nef-associated factor 1, that increases cell surface CD4 expression. FEBS Lett. 442:83-88.

16. Wu (2001) ILK Interactions. J. Cell Sci. 114:2549-2550.

17. Torii etal. (1999) Organization of the human orphan nuclear receptor Nurr1 gene. Gene 230:225-232.

18. Chesi et al. (1998) Frequent dysregulation of the c-maf proto-oncogene at 16q23 by translocation to an 1g locus in multiple myeloma. Blood 91:4457-4463.

19. Tang et al. (2002) The forkhead transcription factor AFX activates apoptosis by induction of the BCL-6 transcriptional repressor J. Biol. Chem. 277:14255-14265.

20. Nishimori etal. (1997) A novel brain-specific p53-target gene, BAI1, containing thrombospondin type 1 repeats inhibits experimental angiogenesis. Oncogene 18:2145-2150.

21. Citron et al. (2002) Protein crosslinking, tissue transglutaminase, alternative splicing and neurodegeneration. Neurochem. Intl. 40:69-78.

22. Kozak (1999) Initiation of translation in prokaryotes and eukaryotes. Gene 234:187-208.

23. Stegh et al. (2000) Identification of the cytolinker plectin as a major early in vivo substrate for caspase 8 during CD95- and tumor necrosis factor receptor-mediated apoptosis. Mol. Cell Biol. 15:5665-5679.

24. Colige et al. (1997) cDNA cloning and expression of bovine procollagen I N-proteinase: a new member of the superfamily of zinc-metalloproteinases with binding sites for cells and other matrix components. Proc. Natl. Acad. Sci. USA 94:2374-2379.

25. Odaka et al. (2000) Nuclear import and subnuclear localization of the proto-oncoprotein ETO (MTG8). Oncogene 19:3584-3597.

26. Deblandre G A, Marinx O P, Evans S S, Majjaj S, Leo O, Caput D, Huez G A, Wathelet M G. Expression cloning of an interferon-inducible 17-kDa membrane protein implicated in the control of cell growth. J. Biol. Chem. 1995;270:23860-23866.

In addition, the expression of other genes was significantly altered in BEC of individuals with Alzheimer's disease as compared to normal aged control (Table 9) (p<0.05): W27720 several genes (+2.3-fold), AL050374 (+2.3-fold), J04164 (−2.3-fold), AC005053 (+2.2-fold), A1557295 (−2.1-fold), and Y11284 (+2.0-fold); CD2 binding protein recognizes the membrane proximal proline rich tandem repeat on the T-cell receptor CD2, which is involved in cytokine production (AF104222; −2.5-fold, p+0.04); fatty acid desaturase (AL050118; −2.2-fold, p=0.046); ecto-ATP dehydrolase I, endothelial CD39/ectoADPase which has a major 30 role in vascular hemostasis by rapidly metabolizing ADP released from platelets, thus preventing further platelet activation and recruitment (AI743406: −2.0-fold, p=0.003); and beta-defensin 2, a cationic peptide crucial component of innate immunity (AF071216; −2.0-fold, p=0.02).

Although changes in some genes did not reach statistical significance in the first U95A analysis using stringent criteria as described, they still may be important for better understanding of defective angiogenesis. For example, semaphorin IlIl which has a role in extension and stabilization of vascular networks was down regulated in MBEC of individuals with Alzheimer's disease by 2-fold in greater than 85% of cases. The gene encoding the Dtk tyrosine kinase receptor, abundantly expressed in differentiating hematopoietic cells, was up regulated by 1.7-fold in greater than 89% of MBEC of individuals with Alzheimer's disease confirming loss of differentiated endothelial phenotype in Alzheimer's disease.

In greater than 92% of Alzheimer's disease cases, the gene encoding the low density lipoprotein receptor related protein-1 (LRP-1) was down regulated by 1.5-fold (p=0.08). LRP-1 activity is down regulated in human senescent endothelial cells in athersclerosis (Vasile et al., FASEB J. 15:458-466, 2001). The LRP-1 protein is down regulated in bran vessels in mice during normal aging and in Alzheimer's disease brains, as we previously reported (Shibata et al., J. Clin. Invest. 106:1489-1499, 2000). Thus, elevated brain Aβ peptide levels could be related to down regulation of LRP-1 in brain endothelial cell (Shibata et al., J. Clin. Invest. 106:1489-1499, 2000) that serves as a clearance receptor for Aβ peptide at the BBB. LRP-1 down regulation could be just a part of a senescent phenotype of Alzheimer's disease brain endothelial cell, but may have a detrimental effect on Aβ accumulation.

Importantly, Aβ40/42 peptides did not produce apoptosis in brain endothelial cell of control or Alzheimer's disease in several assays that we have used, confirming that brain endothelium has no enhanced sensitivity to wild type Aβ peptide as described for other cell types. Thus, endothelial changes that have been observed in these studies may be independent of the presence of Aβ peptide, but the CNS accumulation of Aβ in Alzheimer's disease could be related to an abnormal endothelial phenotype. In support of this hypothesis, we generated new data showing that incubation of normal or AD BEC with Aβ40/Aβ42 at the levels of 25 nM for each peptide does not affect the cell cycle or BEC, and does not affect their angiogenic potential. Increasing concentrations of Aβ40/Aβ42 up to 500 nM were also without effect and did not induce either the senescent phenotype in BEC or altered brain capillary morphogenesis. We also studied BEC isolated from a mouse model of AD, Tg2576sw+/−, with a Swedish APP mutation. BEC isolated from 20 months old mice were not senescent and performed normally in an angiogenesis 2-D gel assay. Thus, our present findings suggest that cerebrovascular amyloidosis could be secondary to primary cell biology and molecular alterations in BEC, rather than affecting BEC biology. These data are consistent with previous reports showing that Aβ does not affect endothelial cells (Miravelle et al., J. Biol. Chem. 275:27110-27116, 2000), in contrast to its detrimental effects on neurons.

In greater than 65% and greater than 69% of all Alzheimer's disease cases, respectively, the multiple drug resistance protein-1 and the ABCA1 protein, the deficit of which is responsible for Tangier disease and familial hypoalphalipoproteinemias, were down regulated by 1.7-fold and 2.6-fold, respectively, thus possibly contributing to brain accumulation of xenobiotics, bile salts, and/or cholesterol in those Alzheimer's disease patients. Down regulation of this gene may reflect cell transformation from a mature differentiated phenotype into a functionally inferior senescent phenotype.

Significant decreases in production and secretion of neurotrophic factors in MBEC derived from Alzheimer's disease patients was shown for nerve growth factor (NGF) by ELISA in cell culture supernatants. Reduced expression of NGF mRNA was confirmed in greater than 80% of Alzheimer's disease cases. This correlated with diminished neuritic outgrowth in hippocampal neurons co-cultured with brain endothelial cell derived from Alzheimer's disease patients. These data are reminiscent of neuritic loss in Alzheimer's disease and an animal model in which intracellular NGF disruption has been achieved (Capsoni et al., Proc. Natl. Acad. Sci. USA 97:6826-6831, 2000).

Additional gene targets were identified in somewhat older AD patients with more severe pathology than were used in our second analysis using Affymetrix U133A and U133B chips (Tables 10-11). BEC from these patients exhibit similar changes in angiogenesis and senescence as seen in FIGS. 1-3, 7 and 9-10. Transcription factor E2F that is involved in apoptosis was increased in AD BEC, as well as cell differentiation factors neuroglin, E74-like factor 4 and neurogranin, while myelin transcription factor 1 was decreased. As shown in the first analysis, the expression of PDE-1 was reduced as well as the expression of another member of the ankyrin. family, ankyrin 1, and some additional cytoskeletal proteins such as myosin VI. in addition to changes in MMP-1, the present analysis showed that MMP-2 is also altered in AD. Both forms of tarnsglutaminase were increased as in the first analysis, while another ribosomal protein poly(rC)-binding protein 3 was decreased. Several genes involved in lipid metabolism, general metabolism and inflammation were also altered, and several forms of potassium channels and transporter for ferritin. A significant number of genes with currently unknown function was altered on both U133A and U133B arrays and listed in Tables 10-11.

The third analysis shows that 181 genes were altered in the same direction in RS-AD BEC and young SIPS BEC, suggesting first, that an AD BEC population with a fully-developed replicative senescent (RS) phenotype (greater than 85%) is very different from an early passage AD BEC population containing only a smaller population of senescent cells (20-40%) and second, that our H₂O₂ model of SIPS in young BEC is very similar to RS-AD BEC, not just in phenotype (i.e., cellular changes) but also in terms of affected genes (Table 12). For example, in both models, a total of 43 cell cycle genes and 19 DNA synthesis. genes were down regulated. In addition, several lysosomal/endosomal genes were up regulated or down regulated suggesting a significant disorder of lysosomes in RS AD BEC and SIPS YC BEC. Staining of AD brains in situ confirmed the expected accumulation of products associated with a lysosomal storage vascular disorder. Expression of adhesion/matrix proteins was increased including several collagens (e.g., collagen IV), which fits well with the increased thickness of the basement membrane in AD (Miyakawa et al., Virchows Arch. 40:121-129, 1982; Yamada, Neuropathology 20:8-22, 2000). Other findings suggest significant comparable abnormalities in cell signaling, and expression of several transcription factors, and genes involved in inflammation, and a number of miscellaneous genes all listed in Table 12.

Each of the altered genes alone and/or in combination with other altered genes in BEC of individuals with Alzheimer's disease found using the Affymetrix U95A, U133A, and U133B arrays, and/or in RS-AD BEC and SIPS YC BEC, could represent a vascular therapeutic target. Correction of a given gene's expression may revert and/or contribute to stabilizing a normal brain endothelial phenotype, which ultimately should result in correction of the cell phenotype.

Altered cellular processes have been identified which can cause defective angiogenesis and an inappropriate senescence. They can be regarded as distinct vascular therapeutic targets in Alzheimer's disease: (1) defective differentiation of brain endothelial cells into vascular tubules when stimulated by growth factors and/or other angiogenic factors; (2) programmed cell death during all stages of angiogenesis and aberrant response to growth factors resulting in activation of programmed cell death via a p53-mediated, p38MAPK-mediated, and/or anioxismediated mechanisms early during cell differentiation; (3) regression in the number of newly formed capillaries and/or vessels due to genomic instability; (4) silent hyperproliferative slow-growing “cancer-like” disease of brain endothelium similar to acute myeloid leukemia caused by the t(8;21)(q22;q22) translocation; (5) disrupted signaling from the plasma membrane to the nucleus; (6) dysregulation of one or more genes which encode transcription factors involved in angiogenesis and differentiation; (7) down regulation of one or more tumor suppressors; (8) abnormal gax-integrin αvβ3 and/or αvβ5 signaling; and (9) over expression of unligated integrins possibly leading to improper signaling through integrin-linked kinase.

Senescence of brain endothelium is of pathogenic and clinical relevance to Alzheimer's vascular disorder and dementia. The present findings also suggest that specific vascular-based prophylactic or therapeutic strategies targeted at presenescent brain endothelial cell can be developed. They can be applied to animal models of Alzheimer's disease, and treatment of patients with Alzheimer's disease or at risk thereof.

The discovery that senescence of cells of the vascular system is implicated in the development of Alzheimer's dementia presents opportunities to implement several strategies using our models: e.g., to provide prophylaxis or therapy in these models including FDA-approved drugs to inhibit growth dysregulation, prevent senescence, assist successful escape from senescence, prevent mitotic catastrophe and/or apoptosis during escape from senescence, etc.

Changes in cellular phenotype and unstable genotype of brain endothelium may result in and/or be associated with senescence and/or mitotic catastrophe which in turn may be caused by a disease-specific Alzheimer's defect in the vascular system. This defect may either reduce the longevity of the vascular system in the brain and/or predispose to stress-induced senescence. The presence of senescent cells in the vascular system can significantly reduce normal physiological functions of the blood-brain barrier related to molecular exchanges between blood and brain, and may impair the cerebral blood flow and alter local intravascular hemostasis. Described herein are (1) replicative senescence of brain endothelial cells in Alzheimer's disease; (2) cell death of brain endothelium through mitotic catastrophe due to inability of cells to enter mitosis; (3) arrest of cells in the G1 phase of the cell cycle and inability of cells to enter the S-phase of the cell cycle; (4) karyokinesis without cytokinesis, or abnormal divisions of cell nuclei without proper cell division leading to accumulation of multinucleated cells; (5) dysregulation of genes encoding key cytoskeletal proteins, translation factors, matrix proteins and transduction signaling; (6) an in vitro model of Alzheimer's like stress-induced premature senescence (SIPS) with sublethal dose of H₂O₂; (7) molecular events leading to SIPS in human brain endothelium including transient elevation of p53 tumor suppressor, lack of Rb phosphorylation, and inhibition of cyclin-dependent kinase inhibitors p21^{cIP1} and p16^{INK4a}; (8) abnormal capillary morphogenesis from senescent cells; (9) potential therapeutic approaches to prevent and/or treat brain endothelial SIPS and mitotic catastrophe.

In addition, a SIPS model of RS AD BEC suitable for testing different drugs has been developed. Studies also suggest significant disorder of lysosomes in senescent cells.

Gene transfer with the candidate genes identified as causing dysregulation of vascular function (e.g., genes listed in Table 9), homeobox gax or mox2, tumor suppressor genes (e.g., gasl, interferon-inducible protein 9-27), transcription factors (e.g., c-MAF, TINUR NGF-B/nurr77), capillary morphogenesis genes (e.g., induction of Sema-3, suppression of BAI1), etc., may be used as described in this invention. Expression constructs can be designed to either produce an increase or decrease in a particular gene product and its cognate pathway as for example, decrease in the case of the gene encoding MTG8-related protein MTG16a. For example, this gene could be regarded as a candidate gene responsible for initiating Alzheimer's disease vascular disorder. Using either constitutive or drug-controlled vectors, expression of key regulatory genes can be used to reverse or attenuate the pathogenic process in the microvessel endothelium. Tissue-specific promoters can be configured into vectors to convey expression to the cell of interest. Repeated application of therapeutic genes is likely to be needed. Following in vitro studies, gene transfer will be performed ex vivo to microvessels and finally in vivo to vessels using different animal models. Such techniques have been successfully applied to endothelial cells.

Antisense, ribozyme, RNA interference, and triple helix strategies may also be used to inhibit the activity of genes which are up regulated (e.g., the gene encoding MTG8-related protein MTG16a).

Similar approaches could be applied for other targets identified with Affymetrix U133A and U133B arrays (Tables 10-11) in other studied cohorts of AD patients and controls (Tables 4-7), or for replicative senescence of BEC AD type and/or SIPS.

High-throughput cell-based assays using fluorescent reporter gene readouts may be developed in several areas for drug screening. Transcription factors that we have discovered to be abnormally regulated can be used in reporter gene constructs (e.g., TINUR NGF-B/nur 77 beta type similar to NOT, c-maf). These factors may either have known cis elements through which they transactivate gene expression or SMB selection can be used to deduce the cis elements when they are previously unknown. Concatenated cis elements may be placed upstream of a fluorescent reporter and this construct stably transfected into mammalian cell lines of several types including those of endothelial or nonendothelial origin, and derived from human or animal species. First-order screening of compounds identifies those compounds that either increase or decrease fluorescence. Second-order screening derive dose-dependent activities for each compound. Third-order screening in our well-characterized cellular models (e.g., MBEC from individuals with Alzheimer's disease) will be followed by in vivo testing in the animal models.

FDA approved anti-neoplastic drugs, including alkylating agents (e.g., cytoxan), nucleoside analogs (e.g., FUdR), or anti-metabolites (e.g., methotrexate) among others, may be used to control abortive cell growth or other drugs used to treat acute myeloid leukemia having t(8;21)(q22;q22) (e.g., doxyrubicin applied at a low dose). These are novel applications for these drugs. Similarly, radiosensitizing agents for vascular delivery and retention in the endothelium may be followed by low level external beam X-irradiation, which is used to control any proliferative disorder leading to dementia (e.g., silent hyperproliferative disorder of brain endothelium). Small therapeutic anti-apoptotic compounds, some of which are FDA approved, may exert anti-apoptotic actions during defective angiogenesis: e.g., SB203580 and SB202190, inhibitors of MAPK p38, molecules that act downstream in the signaling pathway such as NF_{κ}-B inhibitors that are activated by MAPK (e.g., terolidin-thio-pyridine carbomaleate), molecules that destabilize p53 or enhance its degradation, inhibitors of cysteine-dependent aspartate cleaving proteases (e.g., ZVAD-fmk or peptide Asp-Glu-Val-Asp-al) may restore at 10 least some cell functions by inhibiting multiple regulator proteases, which activate the caspase signaling pathway, or a particular effector protease (e.g., caspase 3), etc.

Small molecules that correct for impaired intracellular signaling may also be used, such as those to block MAPK and signals that are induced by phosphorylated MAPK, to increase signaling within the GTP/cGMP pathway, to inhibit increased L-3-phophosertine phosphatase activity, or to increase PDE1 and PDE1B1 activity. Candidate compounds include PD98059, an inhibitor of MAPK. Forskolin may have the potential to reestablish impaired signaling due to down regulation of GTPase activating proteins (REM), to block activated tyrosine kinase receptors, etc. Anti-oxidants such as GSH and N-acetyl-cysteine, or S-adenosyl methionine may alter the redo state of the cell and alleviate apoptotic signals.

All references (e.g., articles, books, patents, and patent applications) cited above are indicative of the level of skill in the art and are incorporated by reference.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. No particular relationship between or among limitations of a claim is meant unless such relationship is explicitly recited in the claim (e.g., the arrangement of components in a product claim or order of steps in a method claim is not a limitation of the claim unless explicitly stated to be so). Thus, all possible combinations and permutations of the individual elements disclosed herein are intended to be considered part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

TABLE 1
Alzheimer's disease patients used for U95A analyses (12,600 genes) to determine a subset of Alzheimer's
disease-specific genes

| | | | | CAUSE OF | VASCULAR | | | | |
|---|---|---|---|---|---|---|---|---|---|
| RAD | AGE | GENDER | PMI | DEATH | RISK FACTORS | ANGIOPATHY | BRAAK | CERAD | CDR |
| 001 | 66 | M | 5 h | Cardiac Arrest | Atherosclerosis | + | V-VI | F | 3 |
| 003 | 86 | F | 3 h | Respiratory | Atherosclerosis | + | V-VI | F | 3 |
| | | | | Failure | | | | | |
| 015 | 67 | F | 4 h 10 m | Respiratory | Atherosclerosis | + | V-VI | M | 4 |
| | | | | Failure | | | | | |
| 20 | 70 | M | 5 h | Pneumonia | None | + | V-VI | M | 4 |
| 36 | 80 | M | 2 h 20 m | Cardiac Arrest | Hypertension | + | V-VI | M | 4 |
| 45 | 66 | F | 4 h | Pneumonia | None | + | V-VI | F | 4 |

TABLE 2
Age-matched controls used for U95A analyses (12,600 genes) to determine a subset of Alzheimer's
disease-specific genes

| | | | | CAUSE OF | VASCULAR | | | | |
|---|---|---|---|---|---|---|---|---|---|
| RAD | AGE | GENDER | PMI | DEATH | RISK FACTORS | ANGIOPATHY | BRAAK | CERAD | CDR |
| 13 | 92 | F | 7 h | Cardiac Arrest | Atherosclerosis | + | 0-I | M | 0.5 |
| 14 | 88 | M | 1 h 24 m | Respiratory | Atherosclerosis | + | 0-I | S | 0 |
| | | | | Arrest | | | | | |
| 16 | 64 | M | 4 h 30 m | Cardiac Arrest | Hypertension, | − | 0-I | 0 | 0 |
| | | | | | Atherosclerosis | | | | |
| 17 | 59 | F | 4 h 30 m | Stroke | None | − | 0 | 0 | 0 |
| 38 | 58 | F | 5 h 30 m | Pulmonary | None | − | 0 | 0 | 0 |
| | | | | Embolism | | | | | |
| 39 | 72 | M | 4 h 20 m | Cardiac Arrest | Atherosclerosis, | − | 0-I | S | 0 |
| | | | | | Myocardial | | | | |
| | | | | | Infarct | | | | |

TABLE 3
Young controls used for U95A analysis (12,600 genes) to determine a
subset of Alzheimer's disease-specific genes

| | | | | CAUSE OF | VASCULAR RISK |
|---|---|---|---|---|---|
| RAD | AGE | GENDER | PMI | DEATH | FACTORS |
| 8 | 37 | F | 6 hrs | Trauma | None |
| 12 | 21 | M | 6 hrs | Trauma | None |
| 18 | 16 | F | 3 h 30 m | Trauma | None |
| 19 | 17 | M | 3 h 30 m | Trauma | None |
| 35 | 26 | M | 3 h 20 m | Trauma | None |

TABLE 4
Alzheimer's disease patients used for U133A and B analyses (45,000 genes) to determine a subset of Alzheimer's
disease-specific genes

| | | | | CAUSE OF | VASCULAR | | | | |
|---|---|---|---|---|---|---|---|---|---|
| RAD | AGE | GENDER | PMI | DEATH | RISK FACTORS | ANGIOPATHY | BRAAK | CERAD | CDR |
| 001 | 66 | M | 5 h | Cardiac Arrest | Atherosclerosis | + | V-VI | F | 3 |
| 002 | 88 | M | 4 h | Sepsis | Atherosclerosis | − | V-VI | F | 3 |
| 003 | 86 | F | 3 h | Respiratory Failure | Atherosclerosis | + | V-VI | F | 3 |
| 015 | 67 | F | 4 h 10 m | Respiratory Failure | Atherosclerosis | + | V-VI | M | 4 |
| 20 | 70 | M | 5 h | Pneumonia | None | + | V-VI | M | 4 |
| 36 | 91 | M | 2 h 20 m | Cardiac Arrest | Hypertension | + | V-VI | M | 4 |
| 037 | 80 | F | 3 h 10 m | Cardiac Arrest | Atherosclerosis | + | V-VI | F | 5 |
| 45 | 66 | F | 4 h | Pneumonia | None | + | V-VI | F | 4 |
| 122 | 99 | F | 3 h 30 m | Cardiac Arrest | Atherosclerosis | + | V-VI | F | 5 |
| 123 | 95 | F | 2 h 30 m | GI Bleeding | None | − | V-VI | F | 3 |
| 124 | 78 | M | 3 h 30 m | Bowel Obstruction | Hypertension | + | V-VI | F | 3 |

TABLE 5
Age-matched controls used for U133A and B analyses (45,000 genes) to determine a subset of Alzheimer's
disease-specific genes

| | | | | CAUSE OF | VASCULAR | | | | |
|---|---|---|---|---|---|---|---|---|---|
| RAD | AGE | GENDER | PMI | DEATH | RISK FACTORS | ANGIOPATHY | BRAAK | CERAD | CDR |
| 13 | 92 | F | 7 h | Cardiac Arrest | Atherosclerosis | + | 0-I | M | 0.5 |
| 14 | 88 | M | 1 h 24 m | Respiratory Arrest | Atherosclerosis | + | 0-I | S | 0 |
| 39 | 72 | M | 4 h 20 m | Cardiac Arrest | Atherosclerosis, | − | 0-I | S | 0 |
| | | | | | Myocardial | | | | |
| | | | | | Infarct | | | | |
| 99 | 74 | F | 2 h 30 m | Lung Carcinoma | Atherosclerosis | + | III-IV | M | 0 |
| 131 | 84 | F | 2 h 45 m | GI Bleeding | None | − | I-II | M | 0 |

TABLE 6
Middle age controls used for U133A and B analyses (45,000 genes) to determine a subset of Alzheimer's
disease-specific genes

| | | | | CAUSE OF | VASCULAR | | | | |
|---|---|---|---|---|---|---|---|---|---|
| RAD | AGE | GENDER | PMI | DEATH | RISK FACTORS | ANGIOPATHY | BRAAK | CERAD | CDR |
| 16 | 64 | M | 4 h 30 m | Cardiac Arrest | Hypertension, | − | 0-I | 0 | 0 |
| | | | | | Atherosclerosis | | | | |
| 17 | 59 | F | 4 h 30 m | Stroke | None | − | 0 | 0 | 0 |
| 38 | 58 | F | 5 h 30 m | Pulmonary | None | − | 0 | 0 | 0 |
| | | | | Embolism | | | | | |
| 67 | 59 | M | 4 h 30 m | Pneumonia | None | − | 0 | S | 0 |
| 127 | 57 | F | 6 h 40 m | Cardiac Arrest | Myocardial | − | 0 | S | 0 |
| | | | | | Infarct | | | | |

TABLE 7
Young controls used for U133A and B analyses (45,000 genes) to
determine a subset of Alzheimer's disease specific genes

| | | | | CAUSE OF | VASCULAR RISK |
|---|---|---|---|---|---|
| RAD | AGE | GENDER | PMI | DEATH | FACTORS |
| 8 | 37 | F | 6 hrs | Trauma | None |
| 18 | 16 | F | 3 h 30 m | Trauma | None |
| 19 | 17 | M | 3 h 30 m | Trauma | None |
| 35 | 26 | M | 3 h 20 m | Trauma | None |
| 77 | 42 | F | 6 hrs | Trauma | None |

TABLE 8
Patients for study of replicative senescence and stress-induced senescence

| | Case | | | PMI | Angi- | | |
|---|---|---|---|---|---|---|---|
| Category | # | Age | Gender | (hr) | opathy | Brakk | CERAD |
| YC | 035 | 26 | M | — | − | ˜ | None |
| YC | 062 | 18 | F | — | − | ˜ | None |
| YC | 063 | 14 | F | — | − | ˜ | None |
| AMC | 013 | 92 | F | 7 | − | ˜ | None |
| AMC | 014 | 88 | M | 1 | − | ˜ | None |
| AMC | 016 | 64 | M | 4.5 | − | 0-I | None |
| AMC | 017 | 59 | F | 4.5 | − | 0 | None |
| AMC | 038 | 58 | F | 5.5 | − | 0 | None |
| AD | 001 | 66 | M | 5 | + | ˜ | Frequent |
| AD | 002 | 88 | M | 4 | − | ˜ | Frequent |
| AD | 003 | 86 | F | 3 | + | V-VI | Frequent |
| AD | 015 | 67 | F | 4.2 | + | V-VI | Moderate |
| AD | 020 | 83 | M | 5 | + | V-VI | Moderate |
| AD | 036 | 91 | M | 2.4 | + | V-VI | Moderate |
| AD | 037 | 80 | F | 3.2 | + | V-VI | Frequent |
| AD | 045 | 66 | F | 4 | − | V-VI | Frequent |

TABLE 9
A subset of Alzheimer's disease-specific genes in AD BEC vs.
age-matched control (AMC) BEC on Affymetrix U95A chips
(12,600 genes). The analysis was based on six AD patients,
six age-matched controls, and five young controls. Details about
patients are given in Tables 1-3. Changes in gene
expression between AD vs. AMC BEC were corrected
for changes in young BEC vs. AMC BEC.

| GenBank | | | |
|---|---|---|---|
| Accession# | Gene Name | Δ Fold | P |
| S77154 | TINUR = NGFI-B/nur77 beta-type | −7.0 | 0.047 |
| | transcription factor | | |
| M55153 | tissue transglutaminase aka TG2 or tTG | 5.8 | 0.00 |
| AJ001612 | L-3-phosphoserine-phosphatase | 5.3 | 0.04 |
| | homologue | | |
| L06499 | ribosomal protein L37a | −3.8 | 0.004 |
| AF055376 | c-maf | −3.1 | 0.005 |
| U40370 | calcium, calmodulin-regulated | −3.0 | 0.015 |
| | 3′,5′-cyclic nucleotide phophodiesterase | | |
| Z54367 | plectin | −3.0 | 0.03 |
| AJ003125 | procollagen I-N proteinase | −2.8 | 0.049 |
| AI381790 | novel adipose specific collagen-like factor | −2.8 | 0.04 |
| X55764 | cytochrome P-45011 beta | 2.5 | 0.005 |
| AF084465 | REM | −2.6 | 0.03 |
| L26336 | human heat shock protein 2 | −2.5 | 0.03 |
| AI011896 | Nef-associated factor 1 beta | 2.5 | 0.007 |
| AF104222 | CD2BP2 CD2 binding protein | −2.5 | 0.04 |
| L19161 | elF-2 gamma | −2.5 | 0.007 |
| X78342 | PISSLRE | 2.5 | 0.01 |
| ??????? | FK506-binding protein | 2.4 | 0.008 |
| X52896 | dermal fibroblast elastin | −2.4 | 0.04 |
| AB005298 | BAI2 homologous to brain-specific | 2.3 | 0.03 |
| | angiogenesis inhibitor 1 | | |
| J04164 | interferon-inducible protein 9-27 | −2.2 | 0.041 |
| Z80776 | histone H2A | 2.2 | 0.041 |
| U13616 | ankyrin G (ANK-3) | −2.2 | 0.03 |
| AJ011311 | AP endonuclease XTH2 | 2.2 | 0.002 |
| U86078 | PDE1B1 calmodulin-stimulated | −2.2 | 0.02 |
| | phosphodiesterase | | |
| AL050118 | fatty acid desaturase 2 (FADS2) | −2.2 | 0.046 |
| AB010419 | MTG8-related protein MTG16a | 2.1 | 0.01 |
| NP039256 | glial growth factor 2 | 2.1 | 0.01 |
| Q02297 | | | |
| AJ133133 | ecto-ATP diphosphohydrolase I | 2.1 | 0.01 |
| AI743406 | GAX | −2.0 | 0.003 |
| AF071216 | beta defensin 2 (HBD2) | −2.0 | 0.02 |
| W27720 | UNKNOWN | 2.3 | 0.05 |
| AL050374 | UNKNOWN | 2.3 | 0.05 |
| J04164 | UNKNOWN | 2.3 | 0.05 |
| AC005053 | whole chromosome | 2.2 | 0.05 |
| AI557295 | unknown (BLASTed)-pancreatic cancer p8 | −2.1 | 0.05 |
| Y11284 | Parkinson's related; distantly related to | 2.0 | 0.05 |
| | Forkhead transcription factor | | |

TABLE 10
Subset of Alzheimer's disease-specific genes in AD BEC vs. age-matched
control (AMC) BEC on Affymetrix U133A chips (22,300 genes). The analysis was
based on 11 AD patients, five age-matched controls, five middle-age controls, and
five young controls. Details about patients and groups are given in Tables 4-7.
Changes in gene expression between AD vs. AMC BEC were corrected for changes
in young BEC vs. AMC BEC. Statistical analysis was performed using Bayesian t-
test (2-fold ratio, signal at 500 expression, and 0.05 Bayesian p-log).

| | | | |
|---|---|---|---|
| Δ Fold | P | Gene Name | GenBank Accession# |

Adhesion

| | | | |
|---|---|---|---|
| −2.06 | 0.043 | osteoblast-specific factor 2 | D13665 |
| | | (fasciclin 1-like) | |

Apoptosis

| | | | |
|---|---|---|---|
| −2.02 | 0.013 | myxovirus (influenza) resistance 1, | NM_002462 |
| | | interferon-inducible protein p78 (mouse) | |

Cell Cycle

| | | | |
|---|---|---|---|
| 2.22 | 0.023 | activator of S phase kinase | NM_006716 |
| 2.17 | 0.023 | origin recognition complex, subunit 1-like | NM_004153 |
| | | (yeast) | |
| 2.25 | 0.008 | MCM4 minichromosome maintenance | AA604621 |
| | | deficient 4 (S. cerevisiae) | |
| 2.40 | 0.008 | H2B histone family, member J | NM_003524 |

Differentiation

| | | | |
|---|---|---|---|
| 3.40 | 0.00005 | neuregulin 1 | NM_013959 |

Immune Response

| | | | |
|---|---|---|---|
| −2.46 | 0.016 | immunoglobulin heavy constant mu | X95660 |
| −2.21 | 0.010 | ribonuclease L (2′,5′-oligoisoadenylate | NM_021133 |
| | | synthetase-dependent) | |

Ion Channels, Transporters

| | | | |
|---|---|---|---|
| 2.12 | 0.043 | potassium intermediate/small | NM_002250 |
| | | conductance calcium-activated | |
| | | channel, subfamily N, member 4 | |
| −2.02 | 0.036 | phospholamban | AW969803 |

Lipid Metabolism

| | | | |
|---|---|---|---|
| 2.18 | 0.009 | prostaglandin E receptor 2 (subtype EP2) | NM_000956 |
| | | 53 kDa | |
| 3.52 | 0.040 | apolipoprotein B mRNA editing enzyme, | NM_004900 |
| | | catalytic polypeptide-like 3B | |
| −2.28 | 0.015 | prostaglanding I2 (prostacyclin) synthase | NM_000961 |

Metabolism

| | | | |
|---|---|---|---|
| 6.01 | 0.002 | transglutaminase 2 (C polypeptide, protein- | BC003551 |
| | | glutamine-gamma-glutamyltransferase) | |
| −2.40 | 0.019 | phenol sulfotransferase | U37025 |
| 2.21 | 0.024 | glycerol kinase | X68285 |
| 2.33 | 0.021 | similar to acetyl-coenzyme A synthetase; | AL049709 |
| | | similar to gamma-glutamyltranspeptidase | |
| 2.11 | 0.008 | glutamate decarboxylase 2 (pancreatic | NM_000818 |
| | | islets and brain, 65 kDa) | |

Matrix

| | | | |
|---|---|---|---|
| 4.03 | 0.037 | matrix metalloproteinase 1 (interstitial | M_002421 |
| | | collagenase) | |

Signal Transduction

| | | | |
|---|---|---|---|
| 2.49 | 0.001 | neuroepithelial cell transforming gene 1 | NM_005863 |
| 2.06 | 0.043 | p21 (CDKN1A)-activated kinase 4 | NM_005884 |
| 3.15 | 0.010 | neurogranin (protein kinase C substrate, | NM_006176 |
| | | RC3) | |
| −2.08 | 0.008 | AMP-activated protein kinase family | NM_014840 |
| | | member 5 | |
| −2.18 | 0.026 | FYN binding protein (FYB-120/130) | AI633888 |
| −2.04 | 0.038 | phosphodiesterase 1A, calmodulin- | NM_005019 |
| | | dependent | |
| 2.04 | 0.034 | ADP-ribosylation factor domain protein 1, | AF230398 |
| | | 64 kDa | |
| 2.22 | 0.014 | G protein-coupled receptor 39 | AL567376 |

Structural, Cytoskeleton

| | | | |
|---|---|---|---|
| −2.41 | 0.040 | Arg/Abl-interacting protein ArgBP2 | NM_021069 |
| −3.28 | 0.027 | ankyrin 1, erythrocytic | NM_000037 |
| 2.43 | 0.010 | myosin VI | U90236 |

Transcription Factors, Regulators

| | | | |
|---|---|---|---|
| 2.23 | 0.001 | E74-like factor 4 (etx domain transcription | NM_001421 |
| | | factor) | |
| 2.65 | 0.026 | E2F transcription factor 1 | NM_005225 |
| −2.26 | 0.011 | myelin transcription factor 1 | M96980 |
| −3.45 | 0.000 | hematopoietic PBX-interacting protein | BP344265 |

Miscellaneous

| | | | |
|---|---|---|---|
| −2.82 | 0.007 | poly(rC) binding protein 3 | NM_020528 |
| | | (RNA-binding) | |
| 2.45 | 0.035 | syntaxin binding protein 2 | AB002559 |
| −2.16 | 0.004 | ferritin H | J04755 |
| −3.29 | 0.002 | peptide transporter 3 | NM_016582 |

Unknown

| | | | |
|---|---|---|---|
| −2.10 | 0.020 | chromosome 22 open reading | BC001292 |
| | | frame 4 | |
| −2.52 | 0.005 | IMAGE: 3460742 | AW170549 |
| −2.12 | 0.041 | hypothetical protein FLJ20699 | NM_017931 |
| −2.20 | 0.008 | secreted protein of unknown | AF173937 |
| | | function | |
| 2.00 | 0.003 | neuropilin (NRP) and tolloid | NM_018092 |
| | | (TLL)-like 2 | |
| −2.33 | 0.005 | LRP16 protein | NM_014067 |
| −2.27 | 0.034 | similar to olfactory receptor, | AA731709 |
| | | family 2; subfamily A, member 4, | |
| | | clone IMAGE: 4424116 | |
| −2.15 | 0.018 | G antigen 3 | NM_001473 |
| 4.05 | 0.021 | hypothetical protein MGC14258 | AV717623 |
| −2.32 | 0.011 | FLJ11412 fis, clone | AK021474 |
| | | HEMBA1000876 | |
| −2.79 | 0.002 | moderately similar to ALU7 | AI820796 |
| −2.01 | 0.041 | weakly similar to POL2_MOUSE | NM_0185751 |
| | | retrovirus-related POL polyprotein | |

TABLE 11
A subset of Alzheimer's disease-specific genes in AD BEC vs. age-
matched control (AMC) BEC on Affymetrix U133B chips (22,300 genes).
The analysis was based on 11 AD patients, five age-matched controls,
five middle-age controls, and five young controls. Details
about patients and groups are given in Tables 4-7.
Changes in gene expression between AD vs. AMC BEC were corrected
for changes in young BEC vs. AMC BEC. Statistical analysis was
performed using Bayesian t-test (2-fold ratio, signal at 500 expression,
and 0.05 Bayesian p-log).

| | | | |
|---|---|---|---|
| | | | GenBank |
| Δ Fold | P | Gene Name | Accession# |

Development

| | | | |
|---|---|---|---|
| −2.07 | 0.016 | limbin | AK234305 |
| 2.18 | 0.010 | sema domain, transmembrane | AK022831 |
| | | domain (TM) and cytoplasmic domain, | |
| | | (semaphorin) 6D | |

Ion Channel, Transporters

| | | | |
|---|---|---|---|
| −2.39 | 0.022 | potassium channel, subfamily K, | AF110523 |
| | | member 7 | |
| 2.11 | 0.023 | adaptor-related protein complex 1, | AA480858 |
| | | sigma 2 subunit | |
| 2.02 | 0.040 | potassium inwardly-rectifying channel, | BF111326 |
| | | subfamily J, member 2 | |
| −2.12 | 0.019 | highly similar to S72269 ryanodine | AA770235 |
| | | receptor isoform 2 | |

Metabolism

| | | | |
|---|---|---|---|
| 2.40 | 0.024 | similar to RIKEN cDNA 2610036L13 | BE614410 |
| 2.28 | 0.038 | splicing factor 3a, subunit 1, 120 kDa | AI655996 |
| −2.60 | 0.009 | weakly similar to activation-induced | AI453548 |
| | | cytidine deaminase | |
| 2.63 | 0.008 | E3 ubiquitin ligase SMURF2 | BF111169 |
| 2.01 | 0.015 | peroxisomal acyl-CoA thioesterase 2B | AA046424 |

Proliferation

| | | | |
|---|---|---|---|
| 2.69 | 0.001 | splicing factor, arginine/serine-rich 3 | BE927772 |

Signal Transduction

| | | | |
|---|---|---|---|
| 2.38 | 0.021 | src family associated phosphoprotein | N21390 |
| 2.21 | 0.030 | 2 serine/threonine kinase 11 | BE671224 |
| | | (Peutz-Jeghers syndrome) | |
| 2.25 | 0.015 | wingless-type MMTV integration site | AW294903 |
| | | family, member 7B | |
| −3.46 | 0.000 | adenylate cyclase 1 (brain) | AK024415 |
| −2.60 | 0.004 | mitogen-activated protein kinase | AK000652 |
| | | phosphatase x | |

Structural, Cytoskeleton

| | | | |
|---|---|---|---|
| −2.18 | 0.040 | Arg/Abl-interacting protein | AI659533 |
| 2.14 | 0.010 | trichohyalin | AI937080 |

Transcription Factors, Regulators

| | | | |
|---|---|---|---|
| 2.15 | 0.009 | MAX gene associated | BF438227 |
| −2.11 | 0.018 | core-binding factor, alpha subunit | NM_004349 |
| | | 2, translocated | |
| 2.38 | 0.035 | MCM10 minichromosome maintenance | AB042719 |
| | | deficient 10 (S. cerevisiae) | |

Zinc Ion Binding

| | | | |
|---|---|---|---|
| −2.34 | 0.010 | tripartite motif-containing 47 | AW249467 |

Unknown

| | | | |
|---|---|---|---|
| 2.29 | 0.010 | hypothetical protein MGC2603 | BC000209 |
| 2.20 | 0.016 | HSPC043 protein | BG391217 |
| −2.53 | 0.005 | PRO1953 | AF130112 |
| 2.51 | 0.007 | hypothetical protein FLJ13456 | N21008 |
| −2.32 | 0.013 | — | AW205640 |
| −2.29 | 0.010 | Homo sapiens cDNA FLJ11862 | AU146128 |
| | | fis, clone HEMBA1006900 | |
| 2.14 | 0.004 | clone RP6-45P1 | AL035397 |
| 2.04 | 0.029 | FLJ30997 fis, clone HLUNG1000104 | BF038869 |
| 2.02 | 0.022 | — | AI445255 |
| 2.35 | 0.041 | — | AW157450 |
| −2.37 | 0.010 | — | AI767250 |
| 2.39 | 0.005 | — | AW291140 |
| −2.31 | 0.005 | — | AW291714 |
| −2.20 | 0.028 | FLJ32757 fis, clone TEST12001766 | AI073559 |
| 2.16 | 0.010 | moderately similar to hypothetical | BF591637 |
| | | protein FLJ20378 | |
| −2.20 | 0.005 | — | AI417160 |
| −2.19 | 0.003 | — | AW511797 |
| 3.91 | 0.001 | — | AW194766 |
| −2.87 | 0.001 | — | AW665538 |

TABLE 12
Changes in gene expression in AD replicative senescence (RS)
BEC and stress-induced premature senescence (SIPS) young control (YC)
BEC on Affymetrix U95A chips. Coincidence analysis revealed a subset of
181 genes altered in the same direction in RS-AD and SIPS-YC (criteria more
greater than 2-fold change, 500 threshold). For SIPS-YC, YC BEG were
treated with H₂O₂ (300 μM) as in FIG. 8, followed by incubation in fresh
medium for three days to develop senescent phenotype. For RS-AD, AD BEC
were cultured for several passages until greater than 85% of cells became
senescent (i.e., β-gal positive, enlarged morphology). SIPS YC BEG (n = 4)
were compared with untreated YC BEC (n = 5) and RS-AD BEC were
compared with early passage AD BEC; n = 3 per group.

| | Acc. # | SIPS-YC | RS-AD |
|---|---|---|---|
| Cell cycle | | | |
| p16INK4 | U26727 | 2.37 | 2.76 |
| growth-arrest-specific protein (gas) | L13720 | 3.83 | 3.22 |
| forkhead box M1 | U74612 | −3.08 | −4.42 |
| p55CDC mRNA | U05340 | −4.76 | −5.17 |
| mitotic checkpoint kinase Mad3L | AF053306 | −3.53 | −5.11 |
| kinesin-like spindle protein HKSP | U37426 | −3.84 | −4.40 |
| kinesin-related protein | D14678 | −2.92 | −2.97 |
| kinesin family member 14 | D26361 | −4.23 | −2.64 |
| mitotic kinesin-like protein-1 | X67155 | −2.09 | −3.53 |
| mitotic centromere-associated kinesin | U63743 | −3.85 | −3.58 |
| lamin B1 | L37747 | −3.14 | −4.57 |
| mitotic checkpoint kinase Bub1 | AF053305 | −3.59 | −4.12 |
| serine/threonine kinase (STK-1) | AF015254 | −2.51 | −5.04 |
| serine/threonine kinase (BTAK) | AF011468 | −3.20 | −4.86 |
| CDC28 protein kinase 1 | AA926959 | −2.63 | −2.14 |
| activator of S phase Kinase | AB028069 | −2.62 | −2.59 |
| CKS1 protein homologue | X54942 | −2.97 | −4.12 |
| CDC2-related protein kinase | M68520 | −2.06 | −2.79 |
| CDC2 | X05360 | −2.71 | −4.26 |
| CDC6-related protein | U77949 | −5.91 | −7.27 |
| cyclin E2 | AF091433 | −2.38 | −2.89 |
| cyclin F | Z36714 | −2.04 | −2.08 |
| cyclin B2 | AL080146 | −2.30 | −5.62 |
| cyclin B | M25753 | −3.86 | −6.32 |
| cyclin A | X51688 | −3.32 | −2.89 |
| Ki-67 antigen | X65550 | −2.63 | −4.86 |
| Mad2 | U65410 | −3.07 | −4.08 |
| MAD2 protein | AJ000186 | −2.17 | −3.23 |
| ZW10 interactor Zwint | AF067656 | −3.81 | −3.94 |
| apoptosis inhibitor surviving | U75285 | −2.59 | −2.50 |
| HOX11L1 gene | AJ002607 | −3.60 | −3.41 |
| B-myb | X13293 | −2.28 | −2.95 |
| centromere-associated protein, CENP-E | Z15005 | −3.42 | −4.37 |
| centromere protein-A (CENP-A) | FU14518 | −3.13 | −3.87 |
| TTK tyrosine kinase | M86699 | −3.02 | −4.26 |
| BRCA1-associated RING domain protein | U76638 | −2.36 | −4.23 |
| polo-like serine/threonine kinase | U01038 | −2.67 | −4.23 |
| fls353 | AB024704 | −3.53 | −3.55 |
| pituitary tumor-transforming 1 | AA203476 | −3.98 | −4.63 |
| barren homolog | D38553 | −2.18 | −2.83 |
| discs, large homolog 7 | D13633 | −2.11 | −5.00 |
| retinoblastoma-associated protein HEC | AF017790 | −3.83 | −4.62 |
| cyclin-selective ubiquitin carrier protein | FU73379 | −4.93 | −7.08 |
| DNA replication/repair | | | |
| DNA topoisomerase ∥ | J04088 | −2.26 | −4.23 |
| topoisomerase ∥ alpha | AI375913 | −2.33 | −5.82 |
| P1-Cdc46 | X74795 | −2.49 | −3.22 |
| P1cdc47 | D55716 | −4.03 | −3.31 |
| thymidylate synthase | X02308 | −2.66 | −3.30 |
| thymidylate synthase | D00596 | −3.18 | −4.40 |
| replication factor C, 37-kDa subunit mRNA | M87339 | −2.32 | −2.97 |
| H2A.X mRNA encoding histone H2A.X | X14850 | −2.49 | −2.80 |
| histone (H2A.Z) | M37583 | −2.05 | −2.38 |
| thymidine kinase | M15205 | −3.63 | −4.84 |
| thymidine kinase | K02581 | −2.85 | −5.04 |
| M1 subunit of ribonucleotide reductase | X59543 | −2.83 | −4.59 |
| chromosome-associated polypeptide-C | AB019987 | −2.24 | −3.16 |
| polymerase (DNA directed), epsilon | AL080203 | −3.06 | −2.70 |
| HMG-2 | X62534 | −2.88 | −3.50 |
| zeste homolog 2 (EZH2) | U61145 | −2.54 | −3.89 |
| ribonuclease H \| large subunit | Z97029 | −3.87 | −3.03 |
| Rad2 nuclease | | −2.21 | −2.19 |
| dihydrofolate reductase | J00140 | −4.08 | −4.05 |
| Lysosomal/endosomal | | | |
| lysosome-associated membrane protein-2 | X77196 | 2.70 | 2.40 |
| lysosomal sialoglycoprotein | D12676 | 2.48 | 2.57 |
| glucosamine-6-sulphatase | Z12173 | 4.02 | 2.81 |
| carboxypeptidase D | U65090 | 2.42 | 2.77 |
| soluble PLA2 receptor | U17034 | 2.49 | 4.38 |
| plasma glutamate carboxypeptidase | AI796048 | 2.72 | 2.03 |
| Cell signaling | | | |
| Ins(1, 3, 4, 5)P4-binding protein | X89399 | 2.40 | 2.74 |
| G alpha subunit | L01694 | 2.69 | 2.37 |
| hR-PTPu gene for protein tyrosine phosphatase | X58288 | 3.04 | 2.45 |
| phospholipase 3D | U60644 | 2.03 | 3.31 |
| Sh3 domain YSC-like 1 | AL050373 | 2.94 | 2.17 |
| GABA(A) receptor-associated protein like 1 | W28281 | 2.20 | 2.13 |
| Ndr protein kinase | Z35102 | 2.44 | 2.04 |
| orphan G protein-coupled receptor HG38 | AF062006 | −2.50 | −2.03 |
| ADP-ribosylation factor (hARF6) | M57763 | −2.98 | −2.08 |
| oncoprotein 18/stathmin | M31303 | −2.59 | −2.70 |
| maternal embryonic leucine zipper kinase (MELK) | D79997 | −2.76 | −3.38 |
| protein tyrosine phosphatase (CIP2) | L25876 | −3.34 | −6.52 |
| Transcriptional regulation | | | |
| transcription elongation factor S-II | D50495 | 2.44 | 2.14 |
| FUS-CHOP protein fusion | S62138 | 4.64 | 3.76 |
| p8 protein | W47047 | 4.30 | 5.51 |
| zinc-finger protein (bcl-6) | U00115 | 2.27 | 2.03 |
| brachyury variant B (TBX1) | AF012131 | 2.01 | 2.61 |
| HOX3D gene for homeoprotein | X61755 | −2.07 | −2.25 |
| ribonucleoprotein H1 | W28483 | −2.35 | −2.39 |
| CDC-like kinase | M59287 | 2.16 | 2.06 |
| leucine-rich acidic nuclear protein like | AA913812 | −2.11 | −2.14 |
| transcription factor RTEF-1 | U63824 | −2.12 | −2.39 |
| transactivator protein (CREB) | M27691 | −2.01 | −2.03 |
| Extracellular matrix/adhesion molecule | | | |
| reversion-inducing-cysteine-rich protein with kazal | AA099265 | 2.39 | 3.80 |
| motifs (RECK) | | | |
| microfibril-associated glycoprotein 4 | L38486 | 5.46 | 13.17 |
| lysyl oxidase (LOX) | L16895 | 2.10 | 2.62 |
| collagenase type IV | M55593 | 3.44 | 2.50 |
| alpha-1 type XVI collagen | M92642 | 2.92 | 3.18 |
| alpha-5 collagen type IV | M58526 | 2.91 | 2.21 |
| integrin binding protein Del-1 | U70312 | 4.29 | 3.40 |
| integrin alpha-2 subunit | X17033 | 3.56 | 2.79 |
| AICL (activation-induced C-type lectin) | X96719 | 2.49 | 2.46 |
| cartilage-associated protein (CASP) | AJ006470 | 2.30 | 2.25 |
| fibulin-5 | AF093118 | 2.45 | 3.59 |
| tetranectin/plasminogen-binding protein | X64559 | 3.00 | 3.84 |
| B-cam/Lutheran blood group glycoproteins | X80026 | −2.31 | −2.16 |
| fibulin-1 B | X53742 | −2.54 | −2.56 |
| intracellular hyaluronic acid binding protein | AF032862 | −3.96 | −4.30 |
| Transporter | | | |
| ATP-binding cassette, sub-family A (ABC1) | AB028985 | 3.16 | 2.51 |
| ATPase, Class V | AI478147 | 2.68 | 2.39 |
| calcium channel alpha-2b subunit | M76559 | 2.70 | 2.31 |
| Na, K-ATPase beta 2 subunit | AF007876 | −2.26 | −2.05 |
| Inflammation | | | |
| TGF-betaIIR alpha | D50683 | 2.60 | 2.07 |
| transforming growth factor-beta 1 binding protein | M34057 | 2.41 | 2.16 |
| latent transforming growth factor-beta binding | | | |
| protein | Z37976 | 2.00 | 3.55 |
| FK506 binding protein 9 | AA487755 | 2.25 | 2.11 |
| importin alpha 7 subunit | AF060543 | −2.38 | −2.05 |
| nuclear localization sequence receptor | | | |
| hSRP1 alpha | U28386 | −2.69 | −3.84 |
| Cell Inflammation | | | |
| immunoglobulin-like transcript 7 | AF041261 | −3.00 | −2.10 |
| Miscellaneous | | | |
| secretogranin ∥ | M25756 | 12.08 | 3.41 |
| heat shock 20-kDa protein | AI093511 | 7.35 | 2.10 |
| semaphorin E | AB000220 | 4.65 | 3.50 |
| propionyl-CoA carboxylase alpha-chain | X14608 | 2.05 | 3.27 |
| hydroxysteroid dehydrogenase | D17793 | 3.18 | 6.02 |
| autotaxin | L35594 | 2.86 | 6.13 |
| spermidine/spermine N1-acetyltransferase | AL050290 | 2.56 | 5.80 |
| glutaminase | AB020645 | 2.33 | 2.26 |
| thioredoxin interacting protein | S73591 | 2.07 | 2.99 |
| beta-mannosidase | U60337 | 2.28 | 3.21 |
| ubiquitin-conjugating enzyme UbcH2 | Z29331 | 3.47 | 4.14 |
| Synaptosomal-associated protein 23B | Y09568 | 2.29 | 2.03 |
| sorting nexin 13 | AB018256 | 2.30 | 2.94 |
| hepatic dihydrodiol dehydrogenase | U05861 | 3.97 | 4.79 |
| DEAD-box protein p72/RNA helicase | U59321 | 3.37 | 3.55 |
| Membrane cofactor protein | X59408 | 3.01 | 2.00 |
| complement factor H | X07523 | 2.22 | 2.70 |
| cleavage signal 1 protein | M61199 | 2.59 | 2.37 |
| Mel-18 protein/zinc finger protein | D13969 | 2.13 | 2.14 |
| amyotrophic lateral sclerosis 2 | AB011121 | 2.01 | 2.04 |
| epithelial membrane protein-2 | U52100 | −2.16 | −2.47 |
| dermatopontin | Z22865 | −2.32 | −2.44 |
| Alstrom syndrome 1 (ALMS1) | R40666 | −2.55 | −2.07 |
| coiled-coil related protein DEEPEST | AF063308 | −2.71 | −4.20 |
| translocase of outer mitochondrial membrane | AF043250 | −2.00 | −2.17 |
| isopeptidase T-3 | U75362 | −2.14 | −2.07 |
| N-acetyltransferase-8 | AB013094 | −2.09 | −2.45 |
| Opa-interacting protein OIP5 | AF025441 | −2.22 | −3.45 |
| HPV16 E1 protein binding protein | U96131 | −2.76 | −3.41 |
| dopamine D2 receptor | X51362 | −3.40 | −2.11 |
| NB thymosin beta | D82345 | −3.06 | −5.32 |
| cytosolic serine hydroxymethyltransferase | L11931 | −2.05 | −3.06 |
| glyceraldehyde-3-phosphate dehydrogenase | U34995 | −11.38 | −2.30 |

1. A method of assaying for vascular dysfunction in a subject affected by or at risk for a neurodegenerative disorder or another cognitive impairment, said method comprising determining whether there is inappropriate senescence and/or defective angiogenesis in at least endothelium of the subject or cells derived from endothelium of the subject.

2. The method according to claim 1, wherein said subject is affected by or at risk for Alzheimer's disease.

3. The method according to claim 1, wherein said subject is human.

4. The method according to claim 1 which further comprises obtaining endothelial cells from said subject and culturing said cells to provide derived cells, wherein inappropriate senescence and/or defective angiogenesis in at least said derived cells is indicative of vascular dysfunction.

5. The method according to claim 1, wherein there is at least (a) abnormal response by endothelial cells to angiogenic signaling; (b) anoikis, apoptosis, or programmed cell death; (c) mitotic catastrophe; (d) a storage disorder, or (e) a combination thereof.

6. The method according to claim 1, wherein there is at least (a) defective differentiation of endothelial cells, (b) defective fusion of capillaries or vessels, (c) inappropriate regression of capillaries or vessels, or (d) a combination thereof.

7. A method of treating a neurodegenerative disorder or another cognitive impairment, said method comprising administration of a drug or other treatment which at least (a) causes neovascularization, (b) reduces defective angiogenesis, (c) reduces defective capillary morphogenesis, (d) reduces senescence, (e) reduces mitotic catastrophe, or (f) a combination thereof in brain and/or vascular system of a subject to provide therapy and/or prevention.

8. A method of treating a neurodegenerative disorder or another cognitive impairment, said method comprising administration of a drug or other treatment which at least (a) reduces vascular dysfunction or (b) normalizes expression of one or more genes whose regulation is dysregulated in brain and/or vascular system of a subject to provide therapy and/or prevention.

9. The method according to claim 7, wherein said neurodegenerative disorder or another cognitive impairment is Alzheimer's disease.

10. The method according to claim 7, wherein said subject is human.

11. The method according to claim 7 further comprising assessing improved cognitive function of said subject after treatment.

12. The method according to claim 7 further comprising monitoring treatment by assessing cerebral blood flow or blood-brain barrier function.

13. A method of determining effectiveness of a drug or other treatment of a neurodegenerative disorder or another cognitive impairment, said method comprising:
(a) applying the drug or other treatment to a subject or an endothelial cell culture;
(b) assaying for vascular dysfunction which is not caused by amyloid; and
(c) selecting said drug or other treatment if there is reversal of vascular dysfunction as potentially effective for said neurodegenerative disorder or another cognitive impairment.

14. The method according to claim 13, wherein at least angiogenesis or senescence is assayed as indicative of said vascular dysfunction.

15. The method according to claim 13, wherein said subject is a nonhuman animal model.

16. The method according to claim 15, wherein said nonhuman animal model is transgenic for a disease-specific gene.

17. The method according to claim 15, wherein said nonhuman animal model has a knock-in or knock-out mutation for a disease-specific gene.

18. A drug or other treatment selected by the method according to claim 13.

19. A method of determining whether cells manifest a phenotype indicative of a neurodegenerative disorder or another cognitive impairment comprising assessing at least (a) angiogenesis, (b) capillary morphogenesis, (c) differentiation of or signal transduction in endothelial cells, (d) DNA damage or repair, (e) mitotic catastrophe, (f) senescence, (g) lysosome function, or (h) a combination thereof.

20. The method according to claim 19, wherein said neurodegenerative disorder or another cognitive impairment is Alzheimer's disease.

21. The method according to claim 19, wherein said cells are derived from a human.

22. The method according to claim 19, wherein said cells are derived from a nonhuman animal.

23. The method according to claim 19 which further comprises selecting a drug or other treatment as potentially effective for therapy and/or prevention by said drug's or other treatment's ability to reverse said phenotype indicative of a neurodegenerative disorder or another cognitive impairment.

24. A drug or other treatment selected by the method according to claim 23.

25. A method of producing stress-induced premature senescent (SIPS) endothelial cells to provide an in vitro model for a neurodegenerative disorder or another cognitive impairment, said method comprising:
(a) providing a culture of endothelial cells,
(b) treating said culture with hydrogen peroxide to induce senescence, and
(c) isolating SIPS endothelial cells from said treated culture.

26. SIPS endothelial cells produced by the method according to claim 25.
